(19) United States
(12) Patent Application Publication
(10) Pub. No.: US 2024/0016805 A1
(43) Pub. Date: Jan. 18, 2024

(54) THERAPEUTIC METHODS AND COMPOSITIONS FOR TREATING PANCREATIC CANCER USING DEVIMISTAT

(71) Applicant: Cornerstone Pharmaceuticals, Inc., Cranbury, NJ (US)

(72) Inventor: Sanjeev Luther, Cranbury, NJ (US)

(21) Appl. No.: 18/251,503

(22) PCT Filed: Nov. 3, 2021
(86) PCT No.: PCT/US2021/057830

§ 371 Date: May 2, 2023

Related U.S. Application Data
(60) Provisional application No. 63/108,929, filed on Nov. 3, 2020.

(51) Int. Cl. A61K 31/513; A61K 9/00; A61P 35/00; A61K 31/20; A61K 31/282; A61K 31/519; A61K 31/4745; A61K 9/08

(52) U.S. Cl. CPC A61K 31/513; A61K 9/0019; A61P 35/00; A61K 31/20; A61K 31/282; A61K 31/519; A61K 31/4745; A61K 9/08

(57) ABSTRACT

The invention provides methods, compositions, and medical kits for treating pancreatic cancer using (i) devimistat in combination with (ii) modified FOLFIRINOX.

### CROSS-REFERENCE TO RELATED APPLICATION

This application claims the benefit of and priority to U.S. Provisional Patent Application Ser. No. 63/108,929, filed Nov. 3, 2020, the contents of which are hereby incorporated by reference in their entirety.

### FIELD OF THE INVENTION

The invention provides methods, compositions, and medical kits for treating pancreatic cancer using (i) devimistat in combination with (ii) modified FOLFIRINOX.

### BACKGROUND

Pancreatic ductal adenocarcinoma (PDA) is the third leading cause of cancer death, and its prognosis is grim, with a 5-year survival rate of 8% (Rahib L, Smith B D, Aizenberg R, Rosenzweig A B, Fleshman J M, Matrisian L M, “Projecting cancer incidence and deaths to 2030: the unexpected burden of thyroid, liver, and pancreas cancers in the United States,” Cancer Research, 2014, 74(11), 2913-21; Siegel R L, Miller K D, Jemal A, “Cancer Statistics, 2017,” CA: a Cancer Journal for Clinicians, 2017, 67(1), 7-30). Gemcitabine (Gemzar®) became the reference regimen for advanced pancreatic cancer for many years, even though gemcitabine offers only marginal improvement in overall survival compared to the prior treatment (5.6 vs. 4.4 months, gemcitabine vs. fluorouracil) (Burris H A, 3rd, Moore M J, Andersen J, Green M R, Rothenberg M L, Modiano M R, et al., “Improvements in survival and clinical benefit with gemcitabine as first-line therapy for patients with advanced pancreas cancer: a randomized trial,” Journal of Clinical Oncology: Official Journal of the American Society of Clinical Oncology, 1997, 15(6), 2403-13; Heinemann V, Boeck S, Hinke A, Labianca R, Louvet C, “Meta-analysis of randomized trials: evaluation of benefit from gemcitabine-based combination chemotherapy applied in advanced pancreatic cancer,” BMC Cancer, 2008, 8, 82). The combination of gemcitabine with a variety of cytotoxic and targeted agents has generally shown no significant survival advantage as compared with gemcitabine alone (Heinemann V, et al, 2008). Recently however, combination gemcitabine and nab-paclitaxel proved beneficial in patients with advanced pancreatic cancer over gemcitabine monotherapy. This combination improved overall (6.7 months to 8.5 months) and progression free survival (PFS, 3.7 months to 5.5 months) in patients with metastatic PDA (Von Hoff D D, Ervin T, Arena F P, Chiorean E G, Infante J, Moore M, et al, “Increased survival in pancreatic cancer with nab-paclitaxel plus gemcitabine,” The New England Journal of Medicine, 2013, 369(18), 1691-703). Promising results have been observed with FOLFIRINOX (a 4-drug combination of 5-FU, leucovorin, irinotecan and oxaliplatin) (Conroy T, Desseigne F, Ychou M, Bouche O, Guimbaud R, Becouarn Y, et al, “FOLFIRINOX versus gemcitabine for metastatic pancreatic cancer,” The New England Journal of Medicine, 2011, 364(19), 1817-25). This combination regimen improved overall survival from 6.8 months with gemcitabine alone, to 11.1 months with the combination. PFS improved from 3.3 months to 6.4 months. The partial response rate increased from 9% to 32%. Notably, there was 1 complete response with FOLFIRINOX out of 171 patients (0.6%). Thus, FOLFIRINOX has become the preferred first-line treatment for patients with advanced PDA at many centers.

Patients included in the phase III study demonstrating superiority of FOLFIRINOX had metastatic pancreatic cancer. However, 25% of all patients with PDA actually present with localized and unresectable disease (Siegel et al. 2017; Winter J M, Brody J R, Abrams R A, Lewis N L, Yeo C J, Cancer of the Pancreas. 10 ed. Baltimore: Lippincott, Williams and Wilkins, 2014). There are therefore limited data on how to treat these patients. Those with stage III PDA (locally advanced) have a slightly improved survival compared to patients with metastatic disease, and they represent a particularly appealing cohort of patients for investigation because there is no direct evidence of spread yet. Patients with stage III or locally advanced PDA cannot undergo a complete surgical resection if they proceed right to surgery due to involvement of major vessels, but with neoadjuvant treatment, a significant proportion of patients (10-20%) become candidates for surgical resection. Definitive surgical therapy may prolong survival even more. In fact, many patients with locally advanced PDA who go on to surgery survive many years (Bickenbach K A, Gonen M, Tang L H, O'Reilly E, Goodman K, Brennan M F, et al, “Downstaging in pancreatic cancer: a matched analysis of patients resected following systemic treatment of initially locally unresectable disease,” Annals of Surgical Oncology, 2012, 19(5), 1663-9).

The locally advanced population is also desirable for larger studies because results may also prove to be applicable to patients with localized and resectable PDA, but who receive neoadjuvant treatment. In both scenarios (stage I, II resectable; as well as stage III unresectable), the main goals of treatment are to contain or shrink the primary, while simultaneously treating occult micrometastatic disease. Many patients with resectable PDA are now receiving neoadjuvant therapy (potentially 25% of all patients with PDA fit into the category of resectable PDA).

At this point in time, standard of care treatment for locally advanced PDA is the same as metastatic PDA, and treatment regimens are extrapolated from the advanced PDA literature (Balaban E P, Mangu P B, Khorana A A, Shah M A, Mukherjee S, Crane C H, et al, “Locally Advanced, Unresectable Pancreatic Cancer: American Society of Clinical Oncology Clinical Practice Guideline,” Journal of Clinical Oncology: Official Journal of the American Society of Clinical Oncology, 2016, 34(22), 2654-68). Radiation has not been proven to improve survival in the locally advanced cohort (Hammel P, Huguet F, van Laethem J L, Goldstein D, Glimelius B, Artru P, et al, “Effect of Chemoradiotherapy vs Chemotherapy on Survival in Patients With Locally Advanced Pancreatic Cancer Controlled After 4 Months of Gemcitabine With or Without Erlotinib: The LAP07 Randomized Clinical Trial,” JAMA: the Journal of the American Medical Association, 2016, 315(17), 1844-53), and is mainly reserved to enhance local control (Balaban, E P et al, 2016). Thus, radiation is not considered part of mandatory standard care for this cohort. Since patients with locally advanced disease may have improved survival compared to those with advanced disease, the PFS from FOLFIRINOX may be slightly better than what had been observed in metastatic trials. However, this is not well investigated. The projected PFS estimate is not known. Small series have reported PFS as high as 10-18 months in well selected groups of patients with locally advanced PDA (Chllamma M K, Cook N, Dhani N C, Giby K, Dodd A, Wang L, et al. “FOLFIRINOX for advanced pancreatic cancer: the Princess Margaret Cancer Centre experience,” British Journal of Cancer, 2016, 115(6), 649-54; Stein S M, James E S, Deng Y, Cong X, Kortmansky J S, Li J, et al, “Final analysis of a phase II study of modified FOLFIRINOX in locally advanced and metastatic pancreatic cancer,” British journal of cancer, 2016, 114(7), 737-43). The PFS appears to be lower in the clinical experience at Thomas Jefferson University, with an overall survival in this group of 14 months (n=69) (Kozak G M, Epstein J D, Deshmukh S P, Scott B B, Keith S W, Lavu H, et al, “Common Hepatic Artery Abutment or Encasement Is an Adverse Prognostic Factor in Patients with Borderline and Unresectable Pancreatic Cancer,” Journal of Gastrointestinal Surgery: Official Journal of the Society for Surgery of the Alimentary Tract, 2017). PFS was not measured in that cohort, but the estimated PFS is historically around 60% of the overall survival, or around 9 months in for the historical cohort with locally advanced PDA (Hammel, P et al, 2016; Chllamma, M K et al, 2016).

The median overall survival of patients with locally advanced PDA in the literature is variably reported because of the heterogeneity of treatments and study group profiles, but we believe that our experience, which is relatively unfiltered (it includes all patients who were treated at Thomas Jefferson University), likely reflects real world data and is about 14 months for patients with borderline resectable or unresectable disease. Indeed, the largest prospective and randomized studies mirror our outcomes; the overall survival was 15 months in the most recent phase III LAP07 trial, as well as the phase II Scalop trial (Hammel, P et al, 2016; Mukherjee S, Hurt C N, Bridgewater J, Falk S, Cummins S, Wasan H, et al, “Gemcitabine based or capecitabine-based chemoradiotherapy for locally advanced pancreatic cancer (SCALOP): a multicentre, randomised, phase 2 trial,” The Lancet Oncology, 2013, 14(4), 317-26).

There is a great medical need for improved therapies to treat patients with locally advanced PDA. The present invention addresses this need and provides other related advantages.

### SUMMARY

The invention provides methods, compositions, and medical kits for treating pancreatic cancer in a patient in need thereof, comprising the step of intravenously administering to the patient:
a. devimistat,
b. oxaliplatin,
c. folinic acid,
d. irinotecan, and
e. fluorouracil
   pursuant to a treatment cycle of 14 days, wherein during the treatment cycle the devimistat is intravenously administered on each of days 1 and 3 at a daily dose of about 500 mg/m², about 750 mg/m², or about 1,000 mg/m², the oxaliplatin is intravenously administered on day 1 as a single dose of about 65 mg/m², the folinic acid is intravenously administered on day 1 as a single dose of about 400 mg/m², the irinotecan is intravenously administered on day 1 as a single dose of about 121 mg/m², and the fluorouracil is intravenously administered on day 1 as a bolus of about 400 mg/m² followed by an infusion of about 2,400 mg/m² for about 42-48 hours over days 1, 2, and 3, in order to treat the pancreatic cancer.

One aspect of the invention provides a method for treating pancreatic cancer in a patient in need thereof, comprising the step of intravenously administering to the patient:
a. devimistat,
b. oxaliplatin,
c. folinic acid,
d. irinotecan, and
e. fluorouracil
   pursuant to a treatment cycle of 14 days, wherein during the treatment cycle the devimistat is intravenously administered on each of days 1 and 3 at a daily dose of about 500 mg/m², about 750 mg/m², or about 1,000 mg/m², the oxaliplatin is intravenously administered on day 1 immediately after the devimistat as a single dose of about 65 mg/m², the folinic acid is intravenously administered on day 1 immediately after the oxaliplatin as a single dose of about 400 mg/m², the irinotecan is intravenously administered on day 1 concurrently with the folinic acid as a single dose of about 121 mg/m², and the fluorouracil is intravenously administered on day 1 immediately after the folinic acid and irinotecan as a bolus of about 400 mg/m² followed by an infusion of about 2,400 mg/m² for about 42-48 hours over days 1, 2, and 3, in order to treat the pancreatic cancer.

Another aspect of the invention provides a method for treating pancreatic cancer in a patient in need thereof, comprising the step of intravenously administering to the patient:
a. devimistat,
b. oxaliplatin,
c. folinic acid,
d. irinotecan, and
e. fluorouracil
   pursuant to a treatment cycle of 14 days, wherein during the treatment cycle the devimistat is administered on each of days 1 and 3 as an IV infusion at the rate of about 4 mL per minute at doses of about 500 mg/m², about 750 mg/m², or about 1,000 mg/m² concurrently with 5% dextrose in water (D5W) at a rate of about 125 mL/hour, the oxaliplatin is administered on day 1 immediately after the devimistat at a dose of about 65 mg/m² as an IV infusion of about two hours, the folinic acid is intravenously administered on day 1 immediately after the oxaliplatin as an infusion of about 90 minutes at a dose of about 400 mg/m², the irinotecan is intravenously administered on day 1 concurrently with the folinic acid at a dose of about 121 mg/m² as an infusion of about 90 minutes via a Y-connector, the fluorouracil is intravenously administered on day 1 immediately after the folinic acid and irinotecan as a bolus of about 400 mg/m² followed by an infusion of about 2,400 mg/m² for about 42-48 hours over days 1, 2, and 3, and the day 3 devimistat dose is intravenously administered either after or concurrently with the fluorouracil, in order to treat the pancreatic cancer.

The foregoing aspects of the invention are described in more detail, along with additional embodiments, in the detailed description below.

### DETAILED DESCRIPTION

### I. Definitions

The term “devimistat” or “CPI-613” refers to 6,8-bis(benzylsulfanyl)octanoic acid, having the chemical structure

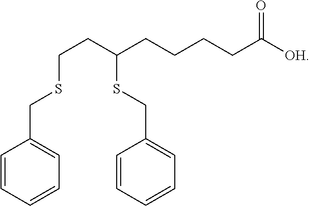 [SMILES: O=C(O)CCCCC(CCSCc1ccccc1)SCc1ccccc1]

As used herein, administering devimistat to a patient includes administering a pharmaceutically acceptable salt form of devimistat to the patient.

The term “folinic acid” or “leucovorin” refers to (4-(((2-amino-5-formyl-4-oxo-1,4,5,6,7,8-hexahydropteridin-6-yl)methyl)amino)benzoyl)-L-glutamic acid, having the chemical structure

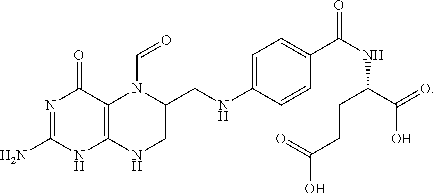 [SMILES: Nc1nc(=O)c2c([nH]1)NCC(CNc1ccc(C(=O)N[C@@H](CCC(=O)O)C(=O)O)cc1)N2C=O]

As used herein, administering folinic acid to a patient includes administering a pharmaceutically acceptable salt form of folinic acid to the patient. When a specific dose of folinic acid is stated in the context of administering folinic acid to a patient, then the amount of pharmaceutically acceptable salt form of folinic acid administered corresponds to the mole amount of folinic acid. For example, the phrase intravenously administering folinic acid as a single dose of about 400 mg/m² (i.e., about 0.84 mmol/m²) encompasses intravenously administering a pharmaceutically acceptable salt of folinic acid (e.g., calcium folinate) at a dose of about 0.84 mmol/m².

The term “irinotecan” refers to (4S)-4,11-diethyl-4-hydroxy-3,14-dioxo-3,4,12,14-tetrahydro-1H-pyrano[3′,4′:6,7]indolizino[1,2-b]quinolin-9-yl 1,4′-bipiperidine-1′-carboxylate, having the chemical structure

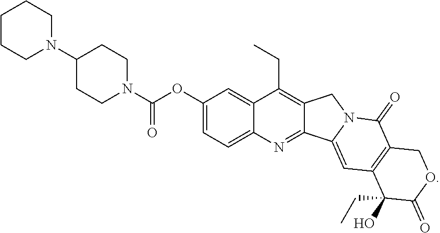 [SMILES: CCc1c2c(nc3ccc(OC(=O)N4CCC(N5CCCCC5)CC4)cc13)-c1cc3c(c(=O)n1C2)COC(=O)[C@]3(O)CC]

As used herein, administering irinotecan to a patient includes administering a pharmaceutically acceptable salt form of irinotecan to the patient. When a specific dose of irinotecan is stated in the context of administering irinotecan to a patient, then the amount of pharmaceutically acceptable salt form of irinotecan administered corresponds to the mole amount of irinotecan. For example, the phrase intravenously administering irinotecan as a single dose of about 121 mg/m² (i.e., about 0.21 mmol/m²) encompasses intravenously administering a pharmaceutically acceptable salt of irinotecan (e.g., irinotecan monohydrochloride, trihydrate) at a dose of about 0.21 mmol/m².

Certain compounds contained in compositions of the present invention may exist in particular geometric or stereoisomeric forms. The present invention contemplates all such compounds, including cis- and trans-isomers, R- and S-enantiomers, diastereomers, (D)-isomers. (L)-isomers, the racemic mixtures thereof, and other mixtures thereof, as falling within the scope of the invention.

As used herein, the term “patient” refers to a human being in need of treatment for pancreatic cancer.

As used herein, the term “treating” includes any effect, e.g., lessening, reducing, modulating, ameliorating or eliminating, that results in the improvement, stabilization, or slowing progression of a condition, disease, disorder, or the like, or a symptom thereof. For example, treatment can include diminishment of a symptom of a disorder or complete eradication of a disorder. As another example, treatment can include slowing the progression of a disease, or preventing or delaying its recurrence, such as maintenance treatment to prevent or delay relapse.

“Therapeutically effective amount” refers to an amount of a compound sufficient to inhibit, halt, or cause an improvement in a disorder or condition being treated in a particular patient or patient population. For example, a therapeutically effective amount can be an amount of drug sufficient to slow the progression of a disease, or to prevent or delay its recurrence, such as maintenance treatment to prevent or delay relapse. A therapeutically effective amount can be determined experimentally in a laboratory or clinical setting, or may be the amount required by the guidelines of the United States Food and Drug Administration, or equivalent foreign agency, for the particular disease and patient being treated. It should be appreciated that determination of proper dosage forms, dosage amounts, and routes of administration is within the level of ordinary skill in the pharmaceutical and medical arts.

As used herein, patients with “locally advanced” disease are patients with invasion of the visceral vessels (mesenteric veins or arteries) such that a complete macroscopic or microscopic resection may not be achieved. These include all localized tumors that are not clearly resectable.

As used herein, locally advanced PDA that is “borderline resectable” is defined with reference to the NCCN guidelines, summarized as follows:
Arterial
   Pancreatic head/uncinate process
      Solid tumor contact with common hepatic artery (CHA) without extension to celiac axis (CA) or hepatic artery bifurcation allowing for safe and complete resection and reconstruction
      Solid tumor contact with the superior mesenteric artery (SMA) of ≤180°
      Solid tumor contact with variant arterial anatomy (ex: accessory right hepatic artery, replaced right hepatic artery, replaced CHA, and the origin of replaced or accessory artery) and the presence and degree of tumor contact should be noted if present, as it may affect surgical planning
   Pancreatic body/tail
      Solid tumor contact with the CA of ≤180°
      Solid tumor contact with the CA of >180° without involvement of the aorta and with intact and uninvolved gastroduodenal artery thereby permitting a modified Appleby procedure (some members prefer this criteria to be in the unresectable category)
   Venous
      Solid tumor contact with the superior mesenteric vein (SMV) or portal vein (PV) of >180°, contact of ≤180° with contour irregularity of the vein or thrombosis of the vein but with suitable vessel proximal and distal to the site of involvement allowing for safe and complete resection and vein reconstruction
      Solid tumor contact with the inferior vena cava (IVC).

In summary, “borderline resectable” PDA distorts the portal or superior mesenteric veins, or abuts visceral arteries (≤180° of the common hepatic artery, celiac trunk, or the superior mesenteric artery).

As used herein, locally advanced PDA that is “unresectable” is defined with reference to the NCCN guidelines, summarized as follows:
Arterial
   Distant metastasis (including non-regional lymph node metastasis)
   Head/uncinate process
      Solid tumor contact with SMA>180°
      Solid tumor contact with the CA>180°
      Solid tumor contact with the first jejunal SMA branch
   Body and tail
      Solid tumor contact of >180° with the SMA or CA
      Solid tumor contact with the CA and aortic involvement
Venous
   Head/uncinate process
      Unreconstructible SMV/PV due to tumor involvement or occlusion (can be due to tumor or bland thrombus)
      Contact with most proximal draining jejunal branch into SMV
   Body and tail
      Unreconstructible SMV/PV due to tumor involvement or occlusion (can be due to tumor or bland thrombus).

In summary, “unresectable” PDA is associated with mesenteric venous occlusion that cannot be reconstructed, or encasement (>180°) of the visceral arteries (SMA, hepatic artery or celiac trunk).

As used herein, the term “pharmaceutical composition” refers to the combination of an active agent (e.g., devimistat) with a pharmaceutically acceptable excipient, suitable for administration to a human being.

The phrase “pharmaceutically acceptable” is employed herein to refer to those compounds, materials, compositions, and/or dosage forms which are, within the scope of sound judgment, suitable for use in contact with the tissues of human beings with acceptable toxicity, irritation, allergic response, and other problems or complications commensurate with a reasonable benefit/risk ratio.

As used herein, the term “pharmaceutically acceptable excipient” refers to any pharmaceutical excipient suitable for use in humans. For examples of such excipients, see e.g., Martin, Remington's Pharmaceutical Sciences, 15th Ed., Mack Publ. Co., Easton, PA [1975].

As used herein, the term “pharmaceutically acceptable salt” refers to any salt (e.g., acid or base) of a compound of the present invention which is suitable for administration to a human being. “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Examples of bases include, but are not limited to, alkali metals (e.g., sodium) hydroxides, alkaline earth metals (e.g., magnesium), hydroxides, ammonia, and compounds of formula NR₃, wherein R is C₁₋₄ alkyl, and the like.

Further examples of salts include salts made using the ion pairing agents described in U.S. Pat. No. 8,263,653, the entire disclosure of which is incorporated by reference herein. Still further ion pairing agents can be selected with guidance from Handbook of Pharmaceutical Salts Properties, Selection and Use, IUPAC, Wiley-VCH, P. H. Stahl, ed., the entire disclosure of which is incorporated by reference herein.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Unless specified otherwise, the term “about” refers to within ±10% of the stated value. The invention encompasses embodiments where the value is within ±9%, ±8%, ±7%, ±6%, ±5%, ±4%, ±3%, ±2%, or ±1% of the stated value.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited steps.

As a general matter, compositions specifying a percentage are by weight unless otherwise specified.

### II. Therapeutic Applications

The invention provides methods, compositions, and medical kits for treating pancreatic cancer in a patient in need thereof, comprising the step of intravenously administering to the patient:
a. devimistat,
b. oxaliplatin,
c. folinic acid,
d. irinotecan, and
e. fluorouracil
   pursuant to a treatment cycle of 14 days, wherein during the treatment cycle the devimistat is intravenously administered on each of days 1 and 3 at a daily dose of about 500 mg/m², about 750 mg/m², or about 1,000 mg/m², the oxaliplatin is intravenously administered on day 1 as a single dose of about 65 mg/m², the folinic acid is intravenously administered on day 1 as a single dose of about 400 mg/m², the irinotecan is intravenously administered on day 1 as a single dose of about 121 mg/m², and the fluorouracil is intravenously administered on day 1 as a bolus of about 400 mg/m² followed by an infusion of about 2,400 mg/m² for about 42-48 hours over days 1, 2, and 3, in order to treat the pancreatic cancer.

One aspect of the invention provides a method for treating pancreatic cancer in a patient in need thereof, comprising the step of intravenously administering to the patient:
a. devimistat,
b. oxaliplatin,
c. folinic acid,
d. irinotecan, and
e. fluorouracil
   pursuant to a treatment cycle of 14 days, wherein during the treatment cycle the devimistat is intravenously administered on each of days 1 and 3 at a daily dose of about 500 mg/m², about 750 mg/m², or about 1,000 mg/m², the oxaliplatin is intravenously administered on day 1 immediately after the devimistat as a single dose of about 65 mg/m², the folinic acid is intravenously administered on day 1 immediately after the oxaliplatin as a single dose of about 400 mg/m², the irinotecan is intravenously administered on day 1 concurrently with the folinic acid as a single dose of about 121 mg/m², and the fluorouracil is intravenously administered on day 1 immediately after the folinic acid and irinotecan as a bolus of about 400 mg/m² followed by an infusion of about 2,400 mg/m² for about 42-48 hours over days 1, 2, and 3, in order to treat the pancreatic cancer.

Another aspect of the invention provides a method for treating pancreatic cancer in a patient in need thereof, comprising the step of intravenously administering to the patient:
a. devimistat,
b. oxaliplatin,
c. folinic acid,
d. irinotecan, and
e. fluorouracil
   pursuant to a treatment cycle of 14 days, wherein during the treatment cycle the devimistat is intravenously administered on each of days 1 and 3 as an IV infusion at the rate of about 4 mL per minute at a daily dose of about 500 mg/m², about 750 mg/m², or about 1,000 mg/m² concurrently with 5% dextrose in water (D5W) at a rate of about 125 mL/hour, the oxaliplatin is intravenously administered on day 1 immediately after the devimistat at a dose of about 65 mg/m² as an IV infusion of about two hours, the folinic acid is intravenously administered on day 1 immediately after the oxaliplatin as an infusion of about 90 minutes at a dose of about 400 mg/m², the irinotecan is intravenously administered on day 1 concurrently with the folinic acid at a dose of about 121 mg/m² as an infusion of about 90 minutes via a Y-connector, the fluorouracil is intravenously administered on day 1 immediately after the folinic acid and irinotecan as a bolus of about 400 mg/m² followed by an infusion of about 2,400 mg/m² for about 42-48 hours over days 1, 2, and 3, and the day 3 devimistat dose is intravenously administered either after or concurrently with the fluorouracil, in order to treat the pancreatic cancer. The method may be further characterized according to one or more features described herein below.

In certain embodiments, the 14-day cycle is repeated at least once. In certain embodiments, the 14-day cycle is repeated at least five times. In certain embodiments, the 14-day cycle is repeated and treatment continues for at least one year. In certain embodiments, the 14-day cycle is repeated and treatment continues until the patient experiences disease progression, inter-current illness that prevents further treatment, or an unacceptable adverse event. In certain embodiments, the 14-day cycle is repeated and treatment continues until the patient experiences disease progression or an unacceptable adverse event.

### Type of Pancreatic Cancer

The method may be further characterized according to the type of pancreatic cancer treated. In certain embodiments, the pancreatic cancer is metastatic pancreatic cancer. In certain embodiments, the pancreatic cancer is locally advanced. In certain embodiments, the pancreatic cancer is locally advanced pancreatic adenocarcinoma. In certain embodiments, the pancreatic cancer is locally advanced pancreatic ductal adenocarcinoma. In certain embodiments, the pancreatic cancer is localized and unresectable (i.e., not considered resectable or removable by a surgeon, without additional treatment) or borderline resectable. In certain embodiments, the pancreatic cancer is histologically or cytologically confirmed pancreatic adenocarcinoma that is locally advanced (including unresectable or borderline resectable). In certain embodiments, the pancreatic cancer is cytologically confirmed pancreatic adenocarcinoma that is locally advanced (including unresectable or borderline resectable) pancreatic cancer based on CT or MRI imaging. In certain embodiments, the pancreatic cancer is histologically or cytologically confirmed pancreatic adenocarcinoma that is locally advanced (including unresectable or borderline resectable) pancreatic cancer based on CT or MRI imaging. In certain embodiments, the pancreatic cancer is histologically or cytologically documented and measurable locally advanced pancreatic adenocarcinoma. In certain embodiments, the pancreatic cancer is previously untreated.

### Devimistat

In the method of the present invention, the devimistat is intravenously administered to the patient in a pharmaceutical composition comprising devimistat and a pharmaceutically acceptable excipient. Any suitable pharmaceutical composition may be used. The devimistat may be formulated using the free acid or a salt thereof.

In certain embodiments, the pharmaceutical composition comprises a pharmaceutically acceptable salt of devimistat and a pharmaceutically acceptable excipient. In certain embodiments, the pharmaceutical composition comprises devimistat and a pharmaceutically acceptable excipient capable of forming an ion pair with devimistat. In certain embodiments, the pharmaceutical composition comprises devimistat and a pharmaceutically acceptable excipient capable of forming an ion pair with devimistat, wherein the pharmaceutically acceptable excipient comprises an aqueous solution of an ion pairing agent. Exemplary ion pairing agents that may be used include, for example, tertiary amines (such as triethanolamine), other amines such as diethanolamine, monoethanolamine, mefenamic acid and tromethamine, and combinations thereof. Other suitable ion pairing agents include organic Bronsted bases, alkali metal hydroxides, and alkaline earth metal hydroxides, such as, for example, cesium hydroxide. Additional exemplary ion pairing agents that may be used to prepare salts of devimistat include, for example, monoalkylamines, dialkylamines, trialkylamines, amino-substituted aliphatic alcohols, hydroxymonoalkylamines, hydroxydialkylamines, hydroxytrialkylamines, amino-substituted heteroaliphatic alcohols, alkyldiamines, substituted alkyldiamines, optionally substituted heteroaryl group containing at least one ring nitrogen atom, polyethyleneimine, polyglutamic acid, ammonia, L-arginine, benethamine benzathine, betaine, calcium hydroxide, choline, deanol, diethanolamine(2,2′-iminobis(ethanol)), diethylamine, 2-(diethylamino)-ethanol, ethanolamine, ethylenediamine, N-methyl-glucamine, hydrabamine, 1H-imidazole, lysine, magnesium hydroxide, 4-(2-hydroxyethyl)-morpholine, piperazine, potassium hydroxide, 1-(2-hydroxyethyl)-pyrrolidine, sodium hydroxide, triethanolamine (2,2′,2″-nitrilotris(ethanol)), tromethamine, and zinc hydroxide. Additional ion pairing agents that may be used to prepare salts of devimistat include diisopropanolamine, 3-amino-1-propanol, meglumine, morpholine, pyridine, niacinamide, tris(hydroxymethyl)aminomethane, 2-((2-dimethylamino)ethoxy)ethanol, 2-(dimethylamino)ethanol, 1-(2-hydroxyethyl)pyrrolidine, and ammonium hydroxide.

In certain embodiments, the pharmaceutical composition comprises devimistat and triethanolamine. In certain embodiments, the pharmaceutical composition comprises devimistat and an aqueous solution of triethanolamine. In certain embodiments, the pharmaceutical composition of devimistat comprises a 50 mg/mL solution of devimistat in 1M aqueous triethanolamine. In certain embodiments, the pharmaceutical composition is diluted with dextrose and water prior to intravenous administration. In certain embodiments, the pharmaceutical composition of devimistat comprises a 50 mg/mL solution of devimistat in 1M aqueous triethanolamine, which is diluted from 50 mg/mL to about 12.5 mg/mL with sterile aqueous 5% dextrose for injection (D5W) prior to administration to the patient. In certain embodiments, the pharmaceutical composition of devimistat comprises a 50 mg/mL solution of devimistat in 1M triethanolamine, and each 1 mL thereof is diluted with about 3 mL D5W prior to administration to a patient. In certain embodiments, the D5W-diluted solution of devimistat has a pH of about 8.4 to about 8.8. Preferably, the D5W-diluted solution of devimistat is stable for at least 24 hours. In certain embodiments, the D5W-diluted devimistat is administered by IV infusion at the rate of about 4 mL/min on day 1 and day 3 of each 14-day cycle. In certain embodiments, the devimistat infusion is administered concurrently with D5W. Preferably, the diluted devimistat pharmaceutical composition is administered concurrently with D5W, wherein the concurrent D5W is administered at the rate of about 125 mL/hour. Preferably, the pharmaceutical composition of devimistat is administered via a central venous catheter. Preferably, the pharmaceutical composition of devimistat is administered via an infusion pump.

In certain embodiments, each devimistat dose is administered intravenously to the patient as a solution containing triethanolamine and about 12.5 mg/mL of devimistat. In certain embodiments, each devimistat dose is administered intravenously to the patient as a solution containing water, triethanolamine, and about 12.5 mg/mL of devimistat. In certain embodiments, each devimistat dose is administered intravenously to the patient as a solution containing dextrose, water, triethanolamine, and about 12.5 mg/mL of devimistat. In certain embodiments, each devimistat dose is administered intravenously to the patient as a solution containing water, about 0.25 M triethanolamine, and about 12.5 mg/mL of devimistat. In certain embodiments, each devimistat dose is administered intravenously to the patient as a solution containing water, dextrose, about 0.25 M triethanolamine, and about 12.5 mg/mL of devimistat. In certain embodiments, each devimistat dose is administered intravenously to the patient as a solution containing triethanolamine and devimistat. In certain embodiments, each devimistat dose is administered intravenously to the patient as a solution containing water, triethanolamine, and devimistat. In certain embodiments, each devimistat dose is administered intravenously to the patient as a solution containing dextrose, water, triethanolamine, and devimistat.

In certain embodiments, the devimistat present in the pharmaceutical composition has a purity of at least about 90% (w/w). In certain embodiments, the devimistat present in the pharmaceutical composition has a purity of at least about 95% (w/w). In certain embodiments, the devimistat present in the pharmaceutical composition has a purity of at least about 98% (w/w). In certain embodiments, the devimistat present in the pharmaceutical composition has a purity of at least about 99% (w/w). In certain embodiments, the devimistat present in the pharmaceutical composition has a purity of at least about 99.3% (w/w). In certain embodiments, the devimistat present in the pharmaceutical composition has a purity of at least about 99.5% (w/w).

The therapeutic method may be further characterized according to the dose of devimistat administered to the patient. The amount of devimistat to be administered is based on the body surface area (BSA) of the patient in square meters (m²). In certain embodiments, the devimistat is intravenously administered to the patient at a dose of about 500 mg/m² to about 1000 mg/m² on each day the devimistat is administered to the patient (i.e., on each of days 1 and 3 of a 14 day cycle). In certain embodiments, the devimistat is intravenously administered to the patient at a dose of about 500 mg/m² on each of days 1 and 3 of a 14 day cycle. In certain embodiments, the devimistat is intravenously administered to the patient at a dose of about 750 mg/m² on each of days 1 and 3 of a 14 day cycle. In certain embodiments, the devimistat is intravenously administered to the patient at a dose of about 1,000 mg/m² on each of days 1 and 3 of a 14 day cycle. In certain embodiments, the devimistat is administered on day 1 of a 14 day cycle prior to administration of modified FOLIRINOX (mFFX), and devimistat is also administered on day 3, after completion of mFFX administration; provided that, in the event that time will not permit devimistat administration following fluorouracil (5-FU) infusion on day 3, the day 3 devimistat dose may be administered concurrently with 5-FU via a double lumen port. The timing of the devimistat dose may be adjusted ±1 day. The devimistat dose may be administered by IV infusion at a rate of IV infusion at a rate of 4 mL/min via a central venous port. The administered dose of devimistat may be adjusted during treatment. For example, the administered dose of devimistat may be reduced if the patient experiences an adverse event. Dose adjustments suitable for use in the method of the present invention are described in Example 1, below.

### Oxaliplatin

In the method of the present invention the oxaliplatin (chemical name cis-[(1R,2R)-1,2 cyclohexanediamine-N,N′] [oxalato(2-)—O,O′] platinum) is intravenously administered to the patient on day 1 of a 14-day cycle as a pharmaceutical composition comprising oxaliplatin and a pharmaceutically excipient. Any suitable pharmaceutical composition may be used. Oxaliplatin is commercially available as a sterile, aqueous solution in 50 mg, 100 mg, and 200 mg vials at a concentration of 5 mg/mL. Preferably, the commercially available solution is used in the method of the present invention. Preferably, the commercially available 5 mg/mL solution is further diluted in an infusion solution of 250-500 mL of 5% dextrose injection (D5W) prior to administration of the diluted solution to the patient.

The amount of oxaliplatin to be administered is based on the body surface area (BSA) of the patient in square meters (m²). The oxaliplatin dose may be administered in accordance with the FDA-approved prescribing information for oxaliplatin. In certain embodiments, the oxaliplatin is intravenously administered to the patient at a dose of about 65 mg/m² on day 1 of a 14 day cycle. Preferably, the oxaliplatin is intravenously administered to the patient at a dose of about 65 mg/m² on day 1 of a 14 day cycle as an infusion of about two hours. Preferably, the oxaliplatin is intravenously administered immediately after administration of the devimistat. The oxaliplatin dose may be administered as a 2-hour IV infusion via a central venous port. The administered dose of oxaliplatin may be adjusted during treatment. For example, the administered dose of oxaliplatin may be reduced if the patient experiences an adverse event. Dose adjustments suitable for use in the method of the present invention are described in Example 1, below.

### Folinic Acid

In the method of the present invention the folinic acid is intravenously administered to the patient on day 1 of a 14-day cycle as a pharmaceutical composition comprising folinic acid and a pharmaceutically acceptable excipient. Any suitable pharmaceutical composition may be used. The folinic acid may be formulated as the free acid or a salt thereof. In certain embodiments, the folinic acid is formulated as an alkaline earth metal salt. In certain embodiments, the folinic acid is formulated as calcium folinate.

Folinic acid is commercially available, such as in 50 mL vials containing a sterile aqueous solution of leucovorin calcium (also known as calcium folinate or calcium N-[p-[[[(6RS)-2-amino-5-formyl-5,6,7,8-tetrahydro-4-hydroxy-6-pteridinyl]methyl]amino]benzoyl]-L-glutamate (1:1)) equivalent to 10 mg/mL leucovorin. Leucovorin calcium is also commercially available as a powder for reconstitution in 50 mg, 100 mg, and 200 mg vials, which when reconstituted with 5 mL, 10 mL, or 20 mL, respectively, of sterile diluent, such as bacteriostatic water for injection or sterile water for injection, contains leucovorin at a concentration of 10 mg/mL (as the calcium salt). Leucovorin calcium is also commercially available in 350 mg vials as a lyophilized powder, which when reconstituted with 17.5 mL of sterile diluent contains leucovorin at a concentration of 20 mg/mL (as the calcium salt). Any of these commercially available solutions may be used in the method of the present invention. Preferably, the commercially available solutions are further diluted in an infusion solution of 5% dextrose injection (D5W), 0.9% NaCl or Ringers solution prior to intravenous administration of the diluted solution to the patient.

The amount of folinic acid to be administered is based on the body surface area (BSA) of the patient in square meters (m²). The folinic acid dose may be administered in accordance with the FDA-approved prescribing information for folinic acid. In certain embodiments, the folinic acid is intravenously administered to the patient at a dose of about 400 mg/m² on day 1 of a 14 day cycle. Preferably, the folinic acid is intravenously administered to the patient on day 1 of a 14 day cycle as an infusion of about 90 minutes at a dose about 400 mg/m². Preferably, the folinic acid is intravenously administered immediately after administration of the oxaliplatin. The folinic acid dose may be administered as a 90-minute IV infusion. The administered dose of folinic acid may be adjusted during treatment. For example, the administered dose of folinic acid may be reduced if the patient experiences an adverse event. Dose adjustments suitable for use in the method of the present invention are described in Example 1, below.

### Irinotecan

In the method of the present invention, the irinotecan is intravenously administered to the patient on day 1 of a 14-day cycle as a pharmaceutical composition comprising irinotecan and a pharmaceutically acceptable excipient. Any suitable pharmaceutical composition may be used. The irinotecan may be formulated as the free base or a salt thereof. In certain embodiments, the irinotecan is formulated as an acid salt. In certain embodiments, the irinotecan is formulated as an HCl salt. In certain embodiments, the irinotecan is formulated as irinotecan monohydrochloride, trihydrate.

Irinotecan monohydrochloride, trihydrate is commercially available as a 20 mg/mL solution in 2 mL and 5 mL vials, which contain about 17.33 mg/mL irinotecan. Preferably, the commercially available solution is used in the method of the present invention. Preferably, the commercially available solution is further diluted in D5W to a final concentration of about 0.12-2.8 mg/mL irinotecan monohydrochloride, trihydrate (equivalent to about 0.10-2.4 mg/mL irinotecan) prior to intravenous administration of the diluted solution to the patient.

The amount of irinotecan to be administered is based on the body surface area (BSA) of the patient in square meters (m²). The irinotecan dose may be administered in accordance with the FDA-approved prescribing information for irinotecan. In certain embodiments, the irinotecan is intravenously administered to the patient at a dose of about 121 mg/m² on day 1 of a 14 day cycle. Preferably, the irinotecan is intravenously administered to the patient at a dose of about 121 mg/m² on day 1 of a 14 day cycle as an infusion of about 90 minutes. In certain embodiments, irinotecan monohydrochloride, trihydrate is intravenously administered to the patient at a dose of about 140 mg/m² on day 1 of a 14 day cycle (equivalent to a dose of about 121 mg/m² irinotecan). Preferably, irinotecan monohydrochloride, trihydrate is intravenously administered to the patient at a dose of about 140 mg/m² on day 1 of a 14 day cycle as an infusion of about 90 minutes (equivalent to a dose of about 121 mg/m² irinotecan).

Preferably, the irinotecan is intravenously administered immediately after administration of the oxaliplatin and concurrently with the folinic acid via a Y-connector. The irinotecan dose may be administered as a 90-minute IV infusion via a central venous port via a Y-connector. The administered dose of irinotecan may be adjusted during treatment. For example, the administered dose of irinotecan may be reduced if the patient experiences an adverse event. Dose adjustments suitable for use in the method of the present invention are described in Example 1, below.

### Fluorouracil

In the method of the present invention the fluorouracil, also known as 5-fluorouracil or 5-FU, chemical name 5-fluoro-1H-pyrimidine-2,4-dione, is intravenously administered to the patient on days 1-3 of a 14-day cycle as a pharmaceutical composition comprising fluorouracil and a pharmaceutically acceptable excipient. Any suitable pharmaceutical composition may be used. Fluorouracil is commercially available as a 50 mg/mL solution in 10 mL, 20 mL, 50 mL, and 100 mL vials. Preferably, the commercially available solution is used in the method of the present invention. For infusion, the commercially available solution is preferably further diluted in D5W to a final concentration of about 2 mg/mL prior to intravenous infusion of the diluted solution to the patient.

The amount of fluorouracil to be administered is based on the body surface area (BSA) of the patient in square meters (m²). The fluorouracil dose may be administered in accordance with the FDA-approved prescribing information for fluorouracil. In certain embodiments, the fluorouracil is intravenously administered to the patient as a bolus of about 400 mg/m² on day 1 of a 14 day cycle, followed by an infusion of about 2.400 mg/m² over about 42-48 hours on days 1-3 of a 14 day cycle. Preferably, the fluorouracil bolus is intravenously administered immediately after administration of the folinic acid and irinotecan. The administered dose of fluorouracil may be adjusted during treatment. For example, the administered dose of fluorouracil may be reduced if the patient experiences an adverse event. Dose adjustments suitable for use in the method of the present invention are described in Example 1, below.

### Pegfilgrastim

In the method of the present invention pegfilgrastim, sold under the tradename NEULASTA®, is optionally administered to the patient following treatment with devimistat and 5FU on day 3 or day 4 of a 14-day cycle as a pharmaceutical composition comprising pegfilgrastim and a pharmaceutically acceptable excipient. Any suitable pharmaceutical composition may be used. Pegfilgrastim is commercially available as a 10 mg/mL solution in in a 0.6 mL prefilled syringe for subcutaneous injection. Preferably, a commercially available composition is used in the method of the present invention. The use of pegfilgrastim should be given as supportive care per NCCN/ASCO guidelines on day 4 or earlier per the treating oncologist (http://ascopubs.org/doi/pdfdirect/10.1200/JC0.2015.62.3488).

The amount of pegfilgrastim to be administered is the same for each patient and is not based on the body surface area (BSA) of the patient. The pegfilgrastim dose may be administered in accordance with the FDA-approved prescribing information for pegfilgrastim.

### Patients for Treatment

Patients treated pursuant to the method of the present invention preferably meet at least one of the following inclusion criteria: a) histologically or cytologically confirmed pancreatic adenocarcinoma; b) locally advanced (including unresectable or borderline resectable) pancreatic cancer; c) Eastern Cooperative Oncology Group (ECOG) Performance status being 0-1 within 2 weeks of planned start of therapy; or d) normal organ and marrow function within 2 weeks prior to treatment. In certain embodiments, patients treated pursuant to the method of the present invention meet at least a) and b) of the above inclusion criteria. In certain embodiments, patients treated pursuant to the method of the present invention meet all of the above inclusion criteria. With respect to inclusion criterion b), the diagnosis of locally advanced, unresectable or borderline resectable, pancreatic cancer may be based on CT or MRI imaging (pancreas protocol CT of the abdomen and pelvis if possible, MRI with contrast or CT with IV contrast in the absence of a pancreas protocol CT scan, CT of the chest with or without contrast), provided that patients with contrast allergies may be permitted without contrast scans for safety reasons. With respect to inclusion criterion d), normal organ and marrow function may be evidenced by: i) adequate hematologic values, ii) adequate hepatic function, iii) adequate renal function, and iv) adequate coagulation. Adequate hematologic values may be evidenced by: A) white blood cell [WBC]≥3500 cells/mm³; B) platelet count ≥100,000 cells/mm³; C) absolute neutrophil count [ANC]≥1500 cells/mm³; and D) hemoglobin ≥8 g/dL. Adequate hepatic function may be evidenced by: A) aspartate aminotransferase [AST/SGOT]≤3× upper normal limit [UNL], B) alanine aminotransferase [ALT/SGPT]≤3×UNL, and C) bilirubin ≤1.5×UNL. Adequate renal function may be evidenced by serum creatinine ≤2.0 mg/dL or 177 μmol/L. Adequate coagulation may be evidenced by International Normalized Ratio (INR) of ≤1.5 unless on therapeutic blood thinners.

In certain embodiments, patients treated pursuant to the method of the present invention do not meet one or more of the following exclusion criteria: a) under 18 years of age; b) endocrine or acinar pancreatic carcinoma; or c) resectable pancreatic cancer. In certain embodiments, patients treated pursuant to the method of the present invention meet none of the above exclusion criteria. In certain embodiments, patients treated pursuant to the method of the present invention meet none of the above exclusion criteria and do not have metastatic pancreatic cancer based on imaging.

### IV. Treatment Efficacy and Safety

The therapeutic method of the present invention may be further characterized by the efficacy and safety of the treatment. Preferably, the method provides an acceptable safety profile, with the benefit of treatment outweighing the risk. When tested in a phase II or phase III clinical trial, the method of the present invention preferably provides an overall survival (OS) of at least three months. In certain embodiments, the phase II or phase III trial is conducted as described in Example 1. Preferably, the phase II or phase III clinical trial comprises at least 30 patients. More preferably, the phase II or phase III clinical trial comprises at least 40 patients. More preferably, the phase II or phase III clinical trial comprises at least 50 patients. More preferably, the phase II or phase III clinical trial comprises at least 75 patients. More preferably, the phase II or phase III clinical trial comprises at least 100 patients. More preferably, the phase II or phase III clinical trial comprises at least 200 patients. More preferably, the phase II or phase III clinical trial comprises at least 300 patients. More preferably, the phase II or phase III clinical trial comprises at least 400 patients. More preferably, the phase II or phase III clinical trial comprises at least 500 patients. Preferably, when tested in such a phase II or phase III clinical trial the method of the present invention provides an OS of at least 14 months. More preferably, the OS is at least 15 months. More preferably, the OS is at least 16 months. More preferably, the OS is at least 17 months. More preferably, the OS is at least 18 months. More preferably, the OS is at least 19 months. More preferably, the OS is at least 20 months. More preferably, the OS is at least 21 months. More preferably, the OS is at least 22 months. More preferably, the OS is at least 23 months. More preferably, the OS is at least 24 months. More preferably, the OS is at least 25 months. More preferably, the OS is at least 26 months. More preferably, the OS is at least 27 months. More preferably, the OS is at least 28 months. More preferably, the OS is at least 29 months. More preferably, the OS is at least 30 months. More preferably, the OS is at least 31 months. More preferably, the OS is at least 32 months. More preferably, the OS is at least 33 months. More preferably, the OS is at least 34 months. Preferably, when tested in such a phase II or phase III clinical trial, the method of the present invention provides an OS longer than the historical OS level observed in previous clinical trials without devimistat. As described in Example 1 below, the inventors have determined that this historical OS level is about 15 months. Preferably, the OS is at least 2 months longer than the historical OS level (i.e., if the historical OS level is 15 months, then the OS of the patients in the clinical trial treated according to the method of the present invention is at least 17 months). More preferably, the OS is at least 3 months longer than the historical OS level. More preferably, the OS is at least 4 months longer than the historical OS level. More preferably, the OS is at least 5 months longer than the historical OS level. More preferably, the OS is at least 6 months longer than the historical OS level. More preferably, the OS is at least 7 months longer than the historical OS level. More preferably, the OS is at least 8 months longer than the historical OS level. More preferably, the OS is at least 9 months longer than the historical OS level. More preferably, the OS is at least 10 months longer than the historical OS level. More preferably, the OS is at least 11 months longer than the historical OS level. More preferably, the OS is at least 12 months longer than the historical OS level. More preferably, the OS is at least 13 months longer than the historical OS level. More preferably, the OS is at least 14 months longer than the historical OS level. More preferably, the OS is at least 15 months longer than the historical OS level. More preferably, the OS is at least 16 months longer than the historical OS level. More preferably, the OS is at least 17 months longer than the historical OS level. More preferably, the OS is at least 18 months longer than the historical OS level.

Preferably, when tested in a phase II or phase III clinical trial as described herein, the method of the present invention provides progression free survival (PFS) of at least 9 months. More preferably, the method of the present invention provides PFS of at least 10 months. More preferably, the PFS is at least 11 months. More preferably, the PFS is at least 12 months. More preferably, the PFS is at least 13 months. More preferably, the PFS is at least 14 months. More preferably, the PFS is at least 15 months. More preferably, the PFS is at least 16 months. More preferably, the PFS is at least 17 months. More preferably, the PFS is at least 18 months. More preferably, the PFS is at least 19 months. More preferably, the PFS is at least 20 months. More preferably, the PFS is at least 21 months. More preferably, the PFS is at least 22 months. More preferably, the PFS is at least 23 months. More preferably, the PFS is at least 24 months. More preferably, the PFS is at least 25 months. More preferably, the PFS is at least 26 months. More preferably, the PFS is at least 27 months. More preferably, the PFS is at least 28 months. Preferably, when tested in a phase II or phase III clinical trial as described herein, the method of the present invention provides a PFS longer than the historical PFS level observed in previous clinical trials without devimistat. As described in Example 1 below, the inventors have determined that this historical PFS level is about 9 months. Preferably, the PFS is at least 1 month longer than the historical PFS level (i.e., if the historical PFS level is 9 months, then the PFS of the patients in the clinical trial treated according to the method of the present invention is at least 10 months). More preferably, the PFS is at least 2 months longer than the historical PFS level. More preferably, the PFS is at least 3 months longer than the historical PFS level. More preferably, the PFS is at least 4 months longer than the historical PFS level. More preferably, the PFS is at least 5 months longer than the historical PFS level. More preferably, the PFS is at least 6 months longer than the historical PFS level. More preferably, the PFS is at least 7 months longer than the historical PFS level. More preferably, the PFS is at least 8 months longer than the historical PFS level. More preferably, the PFS is at least 9 months longer than the historical PFS level. More preferably, the PFS is at least 10 months longer than the historical PFS level. More preferably, the PFS is at least 11 months longer than the historical PFS level. More preferably, the PFS is at least 12 months longer than the historical PFS level. More preferably, the PFS is at least 13 months longer than the historical PFS level. More preferably, the PFS is at least 14 months longer than the historical PFS level. More preferably, the PFS is at least 15 months longer than the historical PFS level. More preferably, the PFS is at least 16 months longer than the historical PFS level. More preferably, the PFS is at least 17 months longer than the historical PFS level. More preferably, the PFS is at least 18 months longer than the historical PFS level.

Preferably, when tested in a phase II or phase III clinical trial as described herein, the percentage of patients who undergo resection after treatment according to the method of the present invention is at least 20%. More preferably, the percentage of patients who undergo resection after treatment according to the method of the present invention is at least 25%. More preferably, the percentage of patients who undergo resection after treatment according to the method of the present invention is at least 30%. More preferably, the percentage of patients who undergo resection after treatment according to the method of the present invention is at least 35%. More preferably, the percentage of patients who undergo resection after treatment according to the method of the present invention is at least 40%. More preferably, the percentage of patients who undergo resection after treatment according to the method of the present invention is at least 45%. More preferably, the percentage of patients who undergo resection after treatment according to the method of the present invention is at least 50%. More preferably, the percentage of patients who undergo resection after treatment according to the method of the present invention is at least 55%. More preferably, the percentage of patients who undergo resection after treatment according to the method of the present invention is at least 60%. More preferably, the percentage of patients who undergo resection after treatment according to the method of the present invention is at least 65%. More preferably, the percentage of patients who undergo resection after treatment according to the method of the present invention is at least 70%. More preferably, the percentage of patients who undergo resection after treatment according to the method of the present invention is at least 75%. More preferably, the percentage of patients who undergo resection after treatment according to the method of the present invention is at least 80%. Preferably, when tested in a phase II or phase III clinical trial as described herein, the percentage of patients who undergo resection after treatment according to the method of the present invention is at least 5% higher than the historical level of patients who undergo resection after treatment in previous clinical trials without devimistat. As described in Example 1 below, the inventors have determined that this historical level of patients who undergo resection after treatment without devimistat is about 20%. Preferably, the percentage of patients who undergo resection after treatment according to the method of the present invention is at least 10% higher than the historical resection rate (i.e., if the historical level of patients who undergo resection after treatment in previous clinical trials without devimistat is 20%, then the percentage of patients who undergo resection after treatment according to the method of the present invention is at least 30%). More preferably, the percentage of patients who undergo resection after treatment according to the method of the present invention is at least 15% higher than the historical resection rate. More preferably, the percentage of patients who undergo resection after treatment according to the method of the present invention is at least 20% higher than the historical resection rate. More preferably, the percentage of patients who undergo resection after treatment according to the method of the present invention is at least 25% higher than the historical resection rate. More preferably, the percentage of patients who undergo resection after treatment according to the method of the present invention is at least 30% higher than the historical resection rate. More preferably, the percentage of patients who undergo resection after treatment according to the method of the present invention is at least 35% higher than the historical resection rate. More preferably, the percentage of patients who undergo resection after treatment according to the method of the present invention is at least 40% higher than the historical resection rate. More preferably, the percentage of patients who undergo resection after treatment according to the method of the present invention is at least 45% higher than the historical resection rate. More preferably, the percentage of patients who undergo resection after treatment according to the method of the present invention is at least 50% higher than the historical resection rate. More preferably, the percentage of patients who undergo resection after treatment according to the method of the present invention is at least 55% higher than the historical resection rate. More preferably, the percentage of patients who undergo resection after treatment according to the method of the present invention is at least 60% higher than the historical resection rate. More preferably, the percentage of patients who undergo resection after treatment according to the method of the present invention is at least 65% higher than the historical resection rate. More preferably, the percentage of patients who undergo resection after treatment according to the method of the present invention is at least 70% higher than the historical resection rate.

Preferably, when tested in a phase II or phase III clinical trial as described herein, the method of the present invention provides time to progression (defined as time from enrollment to progression; TTP) of at least three months. More preferably, the TTP is at least 4 months. More preferably, the TTP is at least 5 months. More preferably, the TTP is at least 6 months. More preferably, the TTP is at least 7 months. More preferably, the TTP is at least 8 months. More preferably, the TTP is at least 9 months. More preferably, the TTP is at least 10 months. More preferably, the TTP is at least 11 months. More preferably, the TTP is at least 12 months. More preferably, the TTP is at least 13 months. More preferably, the TTP is at least 14 months. More preferably, the TTP is at least 15 months. More preferably, the TTP is at least 16 months. More preferably, the TTP is at least 17 months. More preferably, the TTP is at least 18 months. More preferably, the TTP is at least 19 months. More preferably, the TTP is at least 20 months. More preferably, the TTP is at least 21 months. More preferably, the TTP is at least 22 months. More preferably, the TTP is at least 23 months. More preferably, the TTP is at least 24 months. Preferably, when tested in a phase II or phase III clinical trial as described herein, the method of the present invention provides a TTP longer than the historical TTP level observed in previous clinical trials without devimistat. Preferably, the TTP is at least 1 month longer than the historical TTP level (e.g., if the historical TTP level is 5 months, then the TTP of the patients in the clinical trial treated according to the method of the present invention is at least 6 months). More preferably, the TTP is at least 2 months longer than the historical TTP level. More preferably, the TTP is at least 3 months longer than the historical TTP level. More preferably, the TTP is at least 4 months longer than the historical TTP level. More preferably, the TTP is at least months longer than the historical TTP level. More preferably, the TTP is at least 6 months longer than the historical TTP level. More preferably, the TTP is at least 7 months longer than the historical TTP level. More preferably, the TTP is at least 8 months longer than the historical TTP level. More preferably, the TTP is at least 9 months longer than the historical TTP level. More preferably, the TTP is at least 10 months longer than the historical TTP level. More preferably, the TTP is at least 11 months longer than the historical TTP level. More preferably, the TTP is at least 12 months longer than the historical TTP level. More preferably, the TTP is at least 13 months longer than the historical TTP level. More preferably, the TTP is at least 14 months longer than the historical TTP level. More preferably, the TTP is at least 15 months longer than the historical TTP level. More preferably, the TTP is at least 16 months longer than the historical TTP level. More preferably, the TTP is at least 17 months longer than the historical TTP level. More preferably, the TTP is at least 18 months longer than the historical TTP level.

Preferably, when tested in a phase II or phase III clinical trial as described herein, the method of the present invention provides a RECIST version 1.1 response rate, including complete response, partial response, and stable disease (RRR) of at least 10%. More preferably, the RRR is at least 20%. More preferably, the RRR is at least 25%. More preferably, the RRR is at least 30%. More preferably, the RRR is at least 35%. More preferably, the RRR is at least 40%. More preferably, the RRR is at least 45%. More preferably, the RRR is at least 50%. More preferably, the RRR is at least 55%. More preferably, the RRR is at least 60%. More preferably, the RRR is at least 65%. More preferably, the RRR is at least 70%. More preferably, the RRR is at least 75%. More preferably, the RRR is at least 80%. More preferably, the RRR is at least 85%. More preferably, the RRR is at least 90%. More preferably, the RRR is at least 95%. Preferably, when tested in a phase II or phase III clinical trial as described herein, the method of the present invention provides an RRR higher than the historical RRR level observed in previous clinical trials without devimistat. Preferably, the RRR is at least 5% higher than the historical RRR level (e.g., if the historical RRR level is 25%, then the RRR of the patients in the clinical trial treated according to the method of the present invention is at least 30%). More preferably, the RRR is at least 10% higher than the historical RRR level. More preferably, the RRR is at least 15% higher than the historical RRR level. More preferably, the RRR is at least 20% higher than the historical RRR level. More preferably, the RRR is at least 25% higher than the historical RRR level. More preferably, the RRR is at least 30% higher than the historical RRR level. More preferably, the RRR is at least 35% higher than the historical RRR level. More preferably, the RRR is at least 40% higher than the historical RRR level. More preferably, the RRR is at least 45% higher than the historical RRR level. More preferably, the RRR is at least 50% higher than the historical RRR level. More preferably, the RRR is at least 55% higher than the historical RRR level. More preferably, the RRR is at least 60% higher than the historical RRR level. More preferably, the RRR is at least 65% higher than the historical RRR level. More preferably, the RRR is at least 70% higher than the historical RRR level. More preferably, the RRR is at least 75% higher than the historical RRR level. More preferably, the RRR is at least 80% higher than the historical RRR level.

Preferably, when tested in a phase II or phase III clinical trial as described herein, the method of the present invention provides a complete pathologic response rate (CRp) of at least 10%. More preferably, the method of the present invention provides CRp of at least 20%. More preferably, the CRp is at least 25%. More preferably, the CRp is at least 30%. More preferably, the CRp is at least 35%. More preferably, the CRp is at least 40%. More preferably, the CRp is at least 45%. More preferably, the CRp is at least 50%. More preferably, the CRp is at least 55%. More preferably, the CRp is at least 60%. More preferably, the CRp is at least 65%. More preferably, the CRp is at least 70%. More preferably, the CRp is at least 75%. More preferably, the CRp is at least 80%. More preferably, the CRp is at least 85%. More preferably, the CRp is at least 90%. More preferably, the CRp is at least 95%. Preferably, when tested in a phase II or phase III clinical trial as described herein, the method of the present invention provides a CRp higher than the historical CRp level observed in previous clinical trials without devimistat. Preferably, the CRp is at least 5% higher than the historical CRp level (e.g., if the historical CRp level is 25%, then the CRp of the patients in the clinical trial treated according to the method of the present invention is at least 30%). More preferably, the CRp is at least 10% higher than the historical CRp level. More preferably, the CRp is at least 15% higher than the historical CRp level. More preferably, the CRp is at least 20% higher than the historical CRp level. More preferably, the CRp is at least 25% higher than the historical CRp level. More preferably, the CRp is at least 30% higher than the historical CRp level. More preferably, the CRp is at least 35% higher than the historical CRp level. More preferably, the CRp is at least 40% higher than the historical CRp level. More preferably, the CRp is at least 45% higher than the historical CRp level. More preferably, the CRp is at least 50% higher than the historical CRp level. More preferably, the CRp is at least 55% higher than the historical CRp level. More preferably, the CRp is at least 60% higher than the historical CRp level. More preferably, the CRp is at least 65% higher than the historical CRp level. More preferably, the CRp is at least 70% higher than the historical CRp level. More preferably, the CRp is at least 75% higher than the historical CRp level. More preferably, the CRp is at least 80% higher than the historical CRp level.

### V. Medical Kits

Another aspect of the invention provides a medical kit containing a therapeutic agent and/or pharmaceutical composition described herein, along with instructions for using the kit to treat pancreatic cancer according the methods described herein. In certain embodiments, the medical kit comprises (i) devimistat, and (ii) instructions for treating pancreatic cancer in a patient with devimistat and mFOLFIRINOX as described herein.

### VI. Examples

The invention now being generally described, will be more readily understood by reference to the following examples, which are included merely for purposes of illustration of certain aspects and embodiments of the present invention, and are not intended to limit the invention.

### Example 1—Treatment of Locally Advanced Pancreatic Cancer in Adult Human Patients Using a Combination of Devimistat and Modified FOLFIRINOX

### Study Design

This is a single arm, phase II trial, of approximately 33 patients with locally advanced pancreatic cancer. The efficacy and safety of devimistat will be examined in conjunction with standard-of-care mFOLFRINOX, as a first-line therapy. Pretreatment, diagnostic biopsy tissue will be collected when available, and clinical data will be evaluated to determine if the combination results in improved overall survival compared to historical experience. We expect to complete accrual in 24 months, and will analyze data 12 months after accrual completion. The study will be completed 12 months after all study activities are completed.

### Primary Objective

The primary objective of the phase II trial is to determine if devimistat increases overall survival (OS), defined as the interval between enrollment and death, when used in combination with mFOLFIRINOX, in patients with locally advanced pancreatic cancer.

### Secondary Objectives

Secondary outcome measures include:
Measurements of progression free survival (PFS) defined as time from study enrollment to progression or death from any cause;
Time to progression (TTP) defined as time from enrolment to progression;
Surgical resection rates
RECIST version 1.1 response rates including complete response, partial response and stable disease (CR+PR+SD)
Complete pathologic response rates (CRp)
Resection margins.

### Correlative Objectives

Exploratory outcomes will include the importance of genetic abnormalities or other molecular markers from tissue biopsies prior to treatment, or from surgical specimens obtained at resection, as predictive biomarkers of safety or efficacy, and serum devimistat and metabolite levels as markers of toxicity or efficacy.

### Inclusion Criteria

Subjects must meet all of the following inclusion criteria to be eligible for enrollment:
Subjects must have histologically or cytologically confirmed pancreatic adenocarcinoma.
Subjects must have locally advanced (including unresectable or borderline resectable) pancreatic cancer based on CT or MRI imaging (pancreas protocol CT of the abdomen and pelvis if possible, MRI with contrast or CT with IV contrast in the absence of a pancreas protocol CT scan, CT of the chest with or without contrast) as determined by the PI or Co-investigators. Patients with contrast allergies may be permitted without contrast scans if approved by the PI or Co-Investigators for safety reasons.
Male or female subjects age >18 years to 80 years of age.
Eastern Cooperative Oncology Group (ECOG) Performance status being 0-1 within 2 weeks of planned start of therapy.
Subjects must have normal organ and marrow function as defined below ≤2 weeks must be:
   Adequate hematologic (white blood cell [WBC]≥3500 cells/mm³; platelet count ≥100.000 cells/mm³; absolute neutrophil count [ANC]≥1500 cells/mm³; and hemoglobin ≥8 g/dL).
   Adequate hepatic function (aspartate aminotransferase [AST/SGOT]≤3× upper normal limit [UNL], alanine aminotransferase [ALT/SGPT]<3× UNL, bilirubin ≤1.5×UNL).
   Adequate renal function (serum creatinine ≤2.0 mg/dL or 177 μmon).
   Adequate coagulation (“International Normalized Ratio” or INR must be ≤1.5) unless on therapeutic blood thinners.
Expected survival ≥3 months in the view of the PI or investigators.
Women of child-bearing potential (i.e., women who are pre-menopausal or not surgically sterile) must use accepted contraceptive methods (abstinence, intrauterine device [IUD], oral contraceptive or double barrier device) during the study, and must have a negative serum or urine pregnancy test within 1 week prior to treatment initiation.
Fertile men must practice effective contraceptive methods (i.e., surgical sterilization, or a condom used with a spermicide) during the study, unless documentation of infertility exists.
No evidence of clinically significant active infection and no serious infection within the past month requiring hospitalization.
Subjects must have the ability to understand and the willingness to sign a written informed consent document.

### Exclusion Criteria

The presence of any of the following will exclude a subject from study enrollment:
Subjects under the age of 18 or older than 80 years of age.
Subjects with endocrine or acinar pancreatic carcinoma.
Subjects with resectable pancreatic cancer.
Subjects with metastatic pancreatic cancer based on imaging.
Subjects who have received prior radiation therapy, surgical or medical treatment for pancreatic cancer.
Subjects receiving any other standard or investigational treatment for their cancer with a primary goal of improving survival within the past 2 weeks prior to initiation of devimistat treatment.
Pregnant women or breast feeding women, or women of child-bearing potential not using reliable means of contraception are excluded from this study because the teratogenic or abortifacient effects of devimistat is unknown. Because there is an unknown, but potential risk for adverse events in nursing infants secondary to treatment of the mother with devimistat, breastfeeding should be discontinued if the mother is treated with devimistat. These potential risks may also apply to other agents used in this study.
Fertile men unwilling to practice contraceptive methods during the study period.
Subjects with a life expectancy less than 3 months.
Subjects with a serious medical illness that would potentially increase subjects' risk for toxicity.
Subjects with any active uncontrolled bleeding, and any patients with a bleeding diathesis (e.g., active peptic ulcer disease).
Subjects with a history of myocardial infarction that is <3 months prior to registration.
Subjects with uncontrolled intercurrent illness including, but not limited to ongoing or active infection, symptomatic congestive heart failure or coronary artery disease, unstable angina pectoris, cardiac arrhythmia, symptomatic myocardial infarction or psychiatric illness/social situations that would limit compliance with study requirements.
Subjects who are known to be HIV-positive and on combination antiretroviral therapy are ineligible because of the potential for pharmacokinetic interactions with devimistat.

### Treatment Plan

Treatment Regimen Overview

This is an open-label study to treat patients with locally advanced, but not metastatic, pancreatic ductal carcinoma who have not received prior chemotherapy for the diagnosis.

Devimistat and mFOLFIRINOX will be administered to patients as shown in the table below. Briefly, a treatment cycle is 2-weeks, with devimistat given on Days 1 and 3 and mFOLFIRINOX given Day 1 through Day 3 of each treatment cycle.

Devimistat is given as an IV infusion at a rate of 4 mL/min via a central venous catheter, at 500 mg/m². Treatment with devimistat may be adjusted +/−1 day. In the event that time will not permit devimistat administration following 5FU infusion on day 3, devimistat may be administered concurrently with 5FU via a double lumen port.

If mFOLFIRINOX is given on the same day as devimistat, it is given immediately after devimistat administration:
Oxaliplatin at 65 mg/m² given as a 2-hr IV infusion
Folinic acid at a dose of 400 mg/m² (administered as leucovorin calcium) is given as a 90 min IV infusion immediately after oxaliplatin, and concurrently with irinotecan via a Y-connector
Irinotecan is administered as a commercially available monohydrochloride, trihydrate salt at a dose of 140 mg/m² (equivalent to about 121 mg/m² irinotecan), given as a 90-min IV infusion via a Y-connector
5FU at 400 mg/m² as bolus followed by a 46-hr infusion at 2400 mg/m², starting immediately after completion of folinic acid and irinotecan. Prepare fluorouracil in D5W only.

TABLE 1
Administration of FOLFIRINOX and Devimistat in Patients
with Locally Advanced Pancreatic Cancer

| Treatment Cycle | | |
|---|---|---|
| Cycle | Day | Administration of Devimistat and/or FOLFIRINIOX |
| Cycle 1 | Day 1 | Devimistat: 500 mg/m², IV infusion at a rate of 4 mL/min via a central |
| (2 wks) | | venous port |
| | | mFOLFIRNOX (given immediately after devimistat administration): |
| | | Oxaliplatin (Eloxatin) at 65 mg/m² given as a 2-hr IV infusion via a central |
| | | venous port |
| | | Folinic acid at 400 mg/m² (administered as leucovorin calcium) is given as |
| | | a 90-min infusion immediately after oxaliplatin, and concurrently with |
| | | irinotecan monohydrochloride, trihydrate (Camptosar). |
| | | Irniotecan is administered as a commercially available monohydrochloride, |
| | | trihydrate salt at 140 mg/m² (equivalent to about 121 mg/m² irinotecan), |
| | | given as a 90-min IV infusion via a central venous port via a Y-connector. |
| | | Fluorouracil (5FU) at 400 mg/m² as bolus followed by a 46-hr infusion at |
| | | 2400 mg/m², starting immediately after completion of folinic acid and |
| | | irinotecan. NOTE: Prepare fluorouracil in D5W. |
| | Day 2 | mFOLFIRINOX: completing the remaining of the 5FU 46-hr infusion |
| | | starting on Day 1 |
| | Day 3 | mFOLFIRINOX: completing the remaining of the 5FU 46-hr infusion |
| | | starting on Day 1, 2 and disconnect the 5FU pump |
| | | devimistat: IV infusion at a rate of 4 mL/min via a central venous catheter |
| | | after completion of 5FU infusion. In the event that time will not permit |
| | | devimistat administration following 5FU infusion, devimistat may be |
| | | administered concurrently with 5FU via a double lumen port. |
| | Day 3 | If indicated, Neulasta should be given as supportive care per NCCN/ASCO |
| | (following | guidelines on Day 4 or earlier per the treating oncologist |
| | treatment | (http://ascopubs.org/doi/pdfdirect/10.1200/JCO.2015.62.3488). |
| | or Day 4) | |
| | Days 5-14 | No treatment |

| | |
|---|---|
| Cycle 2 | Same as Cycle 1 |
| Cycle 3 | Same as Cycle 1 |
| Etc. | Total: 12 cycles (6 months) |

hr = hour;
IV = intravenous via a central venous port;
min = minutes;
wks = weeks

Upon completion of 12 cycles, or discontinuation of therapy due to toxicity or other reasons, patients may receive additional therapy according to clinicians' recommendation. These treatments may include, but are not limited to additional mFOLFRINOX+devimistat, other chemotherapy regimen, chemoradiation, resection, other clinical trial, or no treatment.

For patients who receive additional treatment with mFOLFIRINOX+devimistat beyond cycle 12, scans should be obtained at least every 4 cycles (or per physician discretion). Patients that continue treatment beyond cycle 12 on either the combination of mFOLFIRINOX+devimistat or devimistat alone will continue to follow the study calendar for each treatment day [i.e. day 1, day 2, day 3 and day 4 for each cycle received beyond cycle 12]. Upon discontinuation of treatment with devimistat patients will have an end of treatment assessment and go into the follow-up period. Patients that continue treatment beyond cycle 12 who are not receiving devimistat will have an end of treatment assessment at the end of cycle 12 and go into the follow-up period. These patients may receive other treatment off study.

The intended duration of therapy is for 12 cycles (2 weeks each) or 6 months. We plan to enroll 33 patients to this trial over a period of 24 months, and investigators and subjects will not be blinded to the treatment. There is no plan for replacement of subjects. Infusion nurses will administer the drug, and research coordinators will be present and available for guidance, according to the protocol.

Study Schedule

The study schedule of events contains the following components:
Pre-Screening/Baseline Period
Treatment Period
Follow up Period

Pre-cycle exams will involve a history and physical to review symptoms. Laboratory tests will be performed. Visits will be at 2 week intervals (±1 day) throughout the duration of the 6 months treatment. There will be no visits for patients withdrawn from the study or who have concluded treatment. For these patients, phone calls will be performed to track follow-up and to administer questionnaires. A final visit will be performed upon withdrawal or completion of the 6 month treatment.

Each biweekly cycle will include chemotherapy infusion, physical exam, history, blood tests (chemistry, CBC, coagulation, CEA, CA 19-9), assessment of adverse events and concomitant medications, and ECOG status. After every 4th cycle during treatment, patients will undergo a CT scan of the chest with or without contrast, abdomen and pelvis with IV contrast, a quality of life questionnaire (FACT-Hep) and have correlative samples collected. After the patients are off treatment, imaging and questionnaires will continue every 3 months for two years. Questionnaires will be performed by phone.

Pre-Study Screening Tests

Pre-study screening tests, which are also enrollment evaluations, must be performed according to the following time frames:

Within 4 weeks: tumor assessments (Pancreas protocol CT of the abdomen and pelvis if possible, MRI with contrast or CT with IV contrast in the absence of a pancreas protocol CT scan, CT of the chest with or without contrast, serum CEA, serum CA 19-9), Tumor Profile Assessment, and optional blood and serum samples.

Within 2 weeks: medical history, physical exam, vital signs, height, weight, ECG, evaluation of symptoms and medications, clinical chemistry, hematology, and coagulation, FACT-Hep quality of life questionnaire.

Within 1 week: pregnancy test for women of child-bearing potential, ECOG.

Safety Assessment

The safety of devimistat and mFOLFIRINOX will be assessed based on: evaluation of symptoms, vital signs, ECOG performance status and survival, clinical chemistry, renal function, hematology, and coagulation.

Safety assessment tests are performed during screening (performed within 2 weeks prior to treatment with devimistat), and prior to each treatment cycle with results available for review within 24 hrs before administration of the anti-tumor agents. CBC with differential and comprehensive metabolic panel (CMP) will be evaluated biweekly, for assessment of potential toxicity of study drugs. ECOG will be performed within 2 weeks of the start of treatment.

These tests are drawn per routine local labs.

Clinical chemistry assessment includes: glucose, creatinine, total protein, albumin, Na⁺, K⁺, Cl⁻, Mg, Ca²⁺, PO₄ (phosphate), CO₂, CA-19-9, CEA, Hemoglobin A1C (drawn every 3 months), BUN, AST/serum glutamic-oxaloacetic transaminase (SCOT), ALT/serum glutamic-pyruvic transaminase (SGPT), alkaline phosphatase (ALP), and total bilirubin.

Hematology includes: complete blood count, differential count, platelet count, hemoglobin, and hematocrit.

Coagulation includes: prothrombin time and partial thromboplastin time.

Tumor Assessment

All patients will be classified prior to treatment as borderline resectable or unresectable, as determined by the PI or Co-investigators, based on the NCCN guideline definitions (see above). The Tumor Profile Assessment must be completed as part of screening potential subjects. Resectable patients are ineligible for the study.

Analyses of Plasma Devimistat Concentrations, Metabolite Concentrations, and Other Analyses.

Plasma samples for the determination of devimistat concentrations, metabolite concentrations and other analyses will be obtained before and at various times after dosing of devimistat in the first 15 patients. This includes:
Day 1 of Cycle 1 at the following time points: −5 min before dosing, and at 0.5, 1, 1.5, 2 & 24 hours after the first dose of devimistat.
Day 3 of Cycle 1, immediately prior to the administration of devimistat.
Day 4 of Cycle 1, immediately prior to the administration of Neulasta, only if Neulasta is given.

Blood (4 mL for each sample) should be obtained from an IV catheter different from the one that is used to administer devimistat. The blood samples should be collected in 4-mL lavender top collection tubes containing K2-EDTA anticoagulant.

Treatment Delays

If patients miss scheduled treatments for reasons other than adverse events and there has not been more than 21 days between treatments, attempts will be made to administer the full 12 cycles unless patients meet a criterion for removal from the study. Follow-up intervals may be adjusted to best sync with treatment administration in the case of delays.

End of Treatment Study Procedures

Final Study Visit (Final Visit, 30 Days after the Final Dose of Study Drug±2 Weeks)
Record adverse events as reported by participant or observed by investigator.
Record results of history and physical examination, vital signs, and ECOG performance status.
Collect blood for clinical lab tests and correlative research samples.
Review post-treatment scan (if ordered).
Complete Quality of Life FACT-Hep Questionnaire

Post-Treatment/Follow-Up

A total treatment course of 6 months (12 cycles) will be attempted for all patients. Blood samples for correlative studies will be collected at 3 months and 6 months following treatment. Patients receiving a clinical benefit but not a candidate for resection will be preferentially maintained on the treatment with devimistat and mFOLFIRINOX. Those patients can have the oxaliplatin omitted at the discretion of the treating investigator and will continue therapy until documented disease progression, ability to undergo surgical resection or the advent of unacceptable toxicity. Since neoadjuvant chemotherapy for resectable disease is considered appropriate standard therapy in many centers to treat occult micrometastatic disease in addition to the primary tumor (Christians K K, Heimler J W, George B, Ritch P S, Erickson B A, Johnston F, et al, “Survival of patients with resectable pancreatic cancer who received neoadjuvant therapy,” Surgery, 2015), a full treatment course of the full 12 cycles is within the standard of care, and prior assessment of resectability will not be performed. Intolerance to chemotherapy is an exception and patients who are deemed surgical candidates but no longer tolerate chemotherapy in the judgement of the treating oncologist may be considered for early resection. Similarly, patients withdrawn from the study may be considered for earlier resection.

All patients will be followed for 2 years from the completion of the study or until death. Patients will not be followed after treatment is completed, with additional research clinic visits. If they decide to continue under the care of the treating oncologist, then imaging will continue every 3 months for 2 years as per standard of care. Alternatively, imaging records will be requested as source documents from other providers. Phone calls will be made every 3 months to obtain follow-up status for 2 years during the surveillance period. Follow-up includes questions regarding disease status and additional treatments including chemotherapy, surgery, and chemoradiation.

General Concomitant Medications and Supportive Care Guidelines

Patients cannot receive any standard or investigational treatment (except devimistat and mFOLFIRINOX) for their cancer, or any other investigational drugs for any indications, while on this study. All concomitant medications (including trade and generic names, dosage and dosing schedule) must be recorded. Concomitant use of anti-emetics is permitted for patients with disease-related nausea.

Criteria for Removal from Study

At least six months of treatment is recommended for patients who have a response or stable disease. Patients should be considered for resection at the completion of this course of chemotherapy. Early termination or consideration of treatment is warranted if:
Patients exhibited disease progression in the opinion of the principal investigator
Unacceptable toxicity from mFOLFIRINOX and/or devimistat
Patient withdrawal of consent
Investigator's discretion to withdraw patients from the study because continued participation in the study is not in the patient's best interest.
Undercurrent illness: a condition, injury, or disease unrelated to the intended disease for which the study is investigating, that renders continuing the treatment unsafe or regular follow-up impossible
General or specific changes in the patient's condition that renders the patient ineligible for further investigational treatment
Non-compliance with investigational treatment, protocol-required evaluations or follow-up visits
Termination of the clinical trial by the sponsor

When terminating treatment during this trial, the investigator should make every effort to contact the patient and to perform a final evaluation with the investigator. Also, the reason(s) for withdrawal from the study must be recorded. After 6 months of treatment or 12 cycles, patients may continue treatment with mFOLFIRINOX and or the study drug until the above criteria are met, and/or may receive other therapies based on the treating oncologist's recommendations. These may include, but are not limited to, resection, radiation, alternative chemotherapy, or stoppage of therapy. Patients who cannot tolerate chemotherapy, may receive any of these alternative therapies prior to the completion of 6 months experimental therapy, including resection, and endpoints will be evaluated in the intent to treat analysis. If patients withdraw from treatment and/or the study, attempts to complete a final visit encounter will be offered, as described above. Intent to treat follow-up information will be attempted for these patients, to collect clinical data through phone calls and other providers to obtain relevant source documentation.

Duration of Follow-Up

Survival and post-study treatment will be documented every three months after the patients complete participation on this trial. All patients will be followed for 2 years from the completion of the study.

For patients who undergo resection, progression after resection will be synonymous with radiographic evidence of recurrence, complemented by other clinical data, and determined by the PI, in consultation with treating physicians (medical oncologist and other surgeons). Progression for all patients will therefore be considered to be an increase in disease burden, as detected for either non-resected or resected patients. OS and other efficacy endpoints will be monitored every 3 months via telephone contact after treatment termination. (Note: During these calls, information related to cancer treatment received after the study will also be collected). OS, TTP, and PFS will be calculated from the date of registration. The duration of OS will be measured until the date of death or censored at last follow-up.

After the patient has received 12 cycles of chemotherapy, radiographic and clinical follow-up will be continued every 3 months until an event has been recorded, or 2 years from the conclusion of treatment. These same objectives will be attempted if feasible for patients receiving additional care at other sites and images and test results requested. Follow-up source information includes physical exam, patient interview (quality of life questionnaire every 3 months for the first year of follow-up), CT chest, abdomen, pelvis with IV contrast (or MRI), and serum CA 19-9 and CEA.

The duration of response (evaluated by PFS) will be measured from the registration date until the first sign of progression assessed by imaging. Progression will be defined according to RECIST version 1.1 criteria or evidence of recurrence after resection (Eisenhauer E A, Therasse P, Bogaerts J, Schwartz L H, Sargent D, Ford R, et al, “New response evaluation criteria in solid tumours: revised RECIST guideline (version 1.1),” Eur J Cancer, 2009, 45(2), 228-47). Comparisons will be made to the initial scan. PFS includes death as an event, while death is censored for TTP.

### Dose Delays/Dose Modifications

Calculation of the Amount of Devimistat for Each Patient

The amount of devimistat at each dose level is based on the BSA of the patient. The BSA values will be calculated based on the height and body weight taken during screening and this BSA value is used throughout the study. This is unless there is a >10% change in the body weight from baseline during the study. At that point, BSA should be revised based on the new body weight and height. The new BSA values will be used from that point on for the remainder of the study, unless there is another >10% change in body weight which will require another revision of the BSA

Supportive treatment may include anti-emetic, anti-diarrhea, anti-pyretic, anti-allergic, anti-hypertensive medications, analgesics, antibiotics, allopurinol, and others such as blood products and bone marrow growth factors. Patients may use erythropoietin for chronic anemia. Also, the hemoglobin should be maintained ≥9 g/dL or ≥90 g/L during the course of the study. The treating physician may utilize erythropoietic factors, or blood or platelet transfusions at their discretion

Dosage Adjustment for Devimistat-Related Toxicities

For adverse events unrelated to serum creatinine elevation or reduction in renal function but possibly related to devimistat, the occurrence of Grade 1 toxicity does not generally require dose modification for subsequent doses for that patient. However, if Grade 2 toxicity (other than alopecia and nausea) probably related to devimistat develops, treatment is to be withheld and can resume only after the Grade 2 toxicity has been reduced to Grade 1 or below, and the dose level for subsequent doses for that patient will be reduced by 25% of the dose (375 mg/m²). Grade 2 alopecia and nausea do not require withholding treatment or dose reduction. If Grade 3 or 4 toxicity probably related to devimistat develops, dosing of devimistat of that patient will be withheld and the patient shall be monitored for recovery from, and reversibility of, such Grade 3 or 4 toxicity. To resume treatment with devimistat for a patient who has had devimistat-related Grade 3 or 4 toxicity, the Grade 3 or 4 toxicity must be reduced to Grade 1 or below, and the dose level for subsequent doses for that patient will be reduced to 50% of the MTD 250 mg/m² (see tables below).

TABLE 2
Dose modifications for non-renal function
AEs related to devimistat

| Dose Level | Devimistat (mg/m²) |
|---|---|
| Study Dose | 500 |
| −1 | 375 |
| −2 | 250 |

TABLE 3
Dose recommendations for non-hematologic AE's,
based on NCI CTCAE Version 5.0, related to devimistat,
and unrelated to renal function*

| Grade | Action |
|---|---|
| 2 (except nausea and | Withhold drug until grade < 2, and resume at |
| alopecia) | next lower dose level. |
| 3 or 4 | Withhold drug until grade < 2, and resume at |
| | the lowest dose. |

*devimistat can only be dropped to −2 doses. The drug will be discontinued with persistent toxicity.

For adverse events related to creatinine elevation or reduction in renal function that are possibly related to devimistat, dosing of the patient will be withheld even if the severity level is Grade 2 or above. Treatment can resume only after the toxicity has been reduced to Grade 1. The dose level for subsequent doses for that patient will be reduced by 15% if the severity level is of Grade 1 (425 mg/m²), by 25% for Grade 2 toxicity (375 mg/m²), and by 50% for Grade 3 or 4 toxicity (250 mg/m²).

TABLE 4
Dose recommendations for non-hematologic AE's,
based on NCI CTCAE Version 5.0, related to
devimistat, and related to renal function*

| Grade | Action |
|---|---|
| 1 | Reduce dose level by 15% from the starting |
| | dose to 425 mg/m². |
| 2 | Withhold drug until grade < 2, and resume at |
| | the next lower dose level. |
| 3 or 4 | Withhold drug until grade < 2, and resume at |
| | the lowest dose. |

*devimistat can only be dropped to −2 doses. The drug will be discontinued with persistent toxicity.

Dosage Adjustment for FOLFIRINOX-Related Toxicities

FOLFIRINOX is a combination of following 4 drugs, all given intravenously (IV) at the following, unmodified, doses in the original phase III trial:
oxaliplatin (Eloxatin) at 85 mg/m² given as a 2-hr IV infusion
folinic acid at 400 mg/m² (administered as leucovorin calcium) is given as a 90-min IV infusion immediately after oxaliplatin
irinotecan is administered as a commercially available monohydrochloride, trihydrate salt at a dose of 180 mg/m² (equivalent to about 156 mg/m² irinotecan), given as a 90-min IV infusion concurrently with folinic acid via a Y-connector
fluorouracil (5FU) at 400 mg/m² as bolus followed by a 46-hr infusion at 2400 mg/m², starting immediately after completion of folinic acid and irinotecan.

However, in clinical practice most of the patients require a dose reduction of the standard FOLFIRINOX due to toxicity. We will use a modified dose FOLFIRINOX upfront (detailed below) to decrease the confounding toxicity of the study combination.

Modified FOLFIRINOX to be Used in this Trial
oxaliplatin (Eloxatin) at 65 mg/m² given as a 2-hr IV infusion
folinic acid at 400 mg/m² (administered as leucovorin calcium) is given as a 90-min infusion immediately after oxaliplatin
irinotecan is administered as a commercially available monohydrochloride, trihydrate salt at 140 mg/m² (equivalent to about 121 mg/m² irinotecan), given as a 90-min IV infusion via a Y connector.
fluorouracil (5FU) at 400 mg/m² as bolus followed by a 46-hr infusion at 2400 mg/m², starting immediately after completion of folinic acid and irinotecan.

The dose of leucovorin is not modified for any toxicity, but is omitted if fluorouracil is omitted. Once a dose of any of the drugs comprising FOLFIRINOX is decreased, reescalation of the dose is not permitted. Patients are taken off the study if they develop the same Grade 4 toxicity despite dose reduction.

Allowable dosing variances for mFOLFIRINOX will be per institutional standards.

The dose adjustment scheme for other drugs of FOLFIRINOX is dependent on the type of toxicities, as described below.

Hematologic Toxicity

Once hematologic toxicity is observed in a patient, do not retreat the patient with FOLFIRINOX until the granulocyte count is ≥1.5×10⁹/L and the platelet count is ≥75×10⁹/L. If a patient experiences neutropenia, prophylactic Neulasta should be added prior to dose reductions outlined below.

The dose modification schemes for FOLFIRINOX related to hematologic toxicity are shown in the table below.]

TABLE 5
Dosages According to the Blood Counts at the
Beginning of a Treatment Cycle (Day 1)

| | Delay the | | | |
|---|---|---|---|---|
| | Start of a | Doses Reduction | | |
| | Treatment | | | Fluorouracil |
| Day 1 | Cycle | Irinotecan | Oxaliplatin | (5FU) |
| Granulocytes < | Withhold | 1st occurrence: | 1st occurrence: | 1st occurrence: |
| 1.5 × 10⁹/L | treatment | reduce the dose | reduce the dose | delete bolus 5FU |
| | until | by 20% | by 20% | |
| | granulocytes ≥ | 2nd occurrence: | 2nd occurrence: | |
| | 1.5 × 10⁹/L | maintain at the | reduce the dose | |
| | | reduced dose | by 20% | |
| | | 3rd occurrence: | 3rd occurrence: | |
| | | reduce the dose | discontinue | |
| | | by 20% | oxaliplatin | |
| | | 4th occurrence: | | |
| | | discontinue | | |
| | | irinotecan | | |
| Platelets < | Withhold | 1st occurrence: | 1st occurrence: | 1st occurrence: |
| 75 × 10⁹/L | treatment | no dose reduction | reduce the dose | reduce both the |
| | until platelets ≥ | | by 20% | bolus and the |
| | 75 × 10⁹/L | | | continuous |
| | | | | infusion dose to |
| | | | | 75% of the |
| | | | | original doses |
| | | 2nd occurrence: | 2nd occurrence: | |
| | | reduce the dose | maintain at the | |
| | | by 20% | reduced | |
| | | 3rd occurrence: | 3rd occurrence: | |
| | | reduce the dose | discontinue | |
| | | by 20% | oxaliplatin | |
| | | 4th occurrence: | | |
| | | discontinue | | |
| | | irinotecan | | |

Consider the use of Filgrastim for recurrent Grade 3/4 neutropenia despite a first-dose reduction or after febrile neutropenia. The use of Neulasta should be given as supportive care per NCCN/ASCO guidelines on day 4 or earlier per the treating oncologist (http://ascopubs.org/doi/pdfdirect/10.1200/JC0.2015.62.3488).

Gastrointestinal Toxicity

Patients must be instructed in the use of loperamide as treatment for diarrhea, and must have a supply of this drug upon starting FOLFIRINOX. Patients should not be retreated with irinotecan until recovery from diarrhea (without loperamide for at least 24 hrs) has occurred. The dose modification scheme related to gastrointestinal toxicity is shown in the table below.

TABLE 6
Dosages According to Gastrointestinal Toxicity

| Adverse Events | Dose Reduction for Subsequent Treatment Cycles |
|---|---|
| Diarrhea Grade 3-4, | 1st occurrence: Reduce the irinotecan dose by 20% and delete |
| or diarrhea + fever | the bolus 5FU dose |
| and/or neutropenia | 2nd occurrence: Reduce also the oxaliplatin dose by 20% and |
| Grade 3-4 | reduce the dose of continuous 5FU to 75% of the original dose |
| | 3rd occurrence: Discontinue treatment |
| Diarrhea ≥ 48 h | No systematic reduction of the irinotecan, oxaliplatin or 5FU |
| despite high doses | doses after complete |
| loperamide | recovery for grade 2 diarrhea. For Grade 3-4 diarrhea, or |
| | diarrhea + fever, and/or concomitant neutropenia Grade 3-4, |
| | reduce chemotherapy as described above |

Mucositis or “Hand-Foot” Syndrome

In case of Grade 3-4 toxicity, a reduction in dosages of 25% of both bolus and continuous 5FU doses in subsequent treatment cycles.

Cardiac Toxicity

In case of angina pectoris or of myocardial infarction, 5FU has to be stopped.

Increase of Bilirubin

In case of bilirubin elevation, it is suggested to exclude an obstruction of the biliary stent or a progressive disease and to postpone chemotherapy. If bilirubin is >1.5× of Upper Limit of Normal (ULN), irinotecan is not recommended. If chemotherapy is medically indicated, it is necessary to provide a dose adjustment of irinotecan.

Neurologic Toxicity

For Grade 2 toxicities that persist between cycles, reduce oxaliplatin by 20%. For any Grade 3 toxicity, reduce the oxaliplatin dose by 20%, and discontinue the drug if symptoms persist between cycles. Discontinue oxaliplatin for any Grade 4 peripheral sensory neuropathy (see table below).

TABLE 7
Dosages According to Neuropathy

| Toxicity | 1-14 Days | Between Cycles |
|---|---|---|
| 2 | — | Reduce oxaliplatin by 20% |
| 3 | Reduce oxaliplatin by 20% | Discontinue |
| 4 | Discontinue | Discontinue |

Other Toxicities

Any other toxicity ≥Grade 2, except anemia and alopecia, can justify a reduction of dose if medically indicated. For example, reduction of irinotecan by 20% and/or oxaliplatin by 20% and/or 5FU of 25% depending of the type of adverse event.

Dietary Restrictions

None.

### Adverse Events and Potential Risks

Devimistat

In the 18 patients who received the MTD in the pilot Phase 1 study (1), common grade ¾ toxicities include: hypokalemia, diarrhea, abdominal pain, hyperglycemia, peripheral neuropathy, neutropenia, lymphopenia, anemia, and thrombocytopenia. Grade 1-2 sensory neuropathy was also observed. Thus, there are no specific risks that have been definitively observed and attributed to devimistat, beyond the toxicities associated with mFOLFIRINOX. The risks of FOLFIRINOX has been well established and include bone marrow suppression, diarrhea, vomiting, fatigue and peripheral sensory neuropathy (Conroy, T et al., 2011; Stein, S M et al., 2016; each cited above).

Possibly related: Alkaline phosphatase, Anorexia, ALT (SGPT), AST (SGOT), Bilirubin (hyperbilirubinemia), Calcium (hypercalcemia, hypocalcemia), *Diarrhea, Flushing, Hemoglobin (anemia), *Injection site Reaction, Leukocytes, Lymphopenia, *Nausea, Neutrophils (neutropenia), Platelets (thrombocytopenia), Potassium, Sodium, *Vomiting, Abnormal heart rate (Asterisk (*) denotes expected Adverse Events).

Probably related: Creatinine (expected Adverse Event).

mFOLFIRINOX

Definitely related: Neutropenia, Febrile neutropenia, Thrombocytopenia, Anemia, Fatigue, Vomiting, Diarrhea, Sensory neuropathy.

Possibly related: Elevated level of alanine aminotransferase.

### Definitions

Adverse Event

An adverse event (AE) is any unfavorable or unintended event, physical or psychological, associated with a research study, which causes harm or injury to a research participant as a result of the participant's involvement in a research study. The event can include abnormal laboratory findings, symptoms, or disease associated with the research study. The event does not necessarily have to have a causal relationship with the research, any risk associated with the research, the research intervention, or the research assessments.

Adverse events may be the result of the interventions and interactions used in the research; the collection of identifiable private information in the research; an underlying disease, disorder, or condition of the subject; and/or other circumstances unrelated to the research or any underlying disease, disorder, or condition of the subject.

Serious Adverse Events

A serious adverse event (SAE) is any adverse experience occurring at any dose that results in any of the following outcomes:
Results in death.
Is a life-threatening adverse experience. The term life-threatening in the definition of serious refers to an adverse event in which the subject was at risk of death at the time of the event. It does not refer to an adverse event which hypothetically might have caused death if it were more severe.
Requires inpatient hospitalization or prolongation of existing hospitalization. Any adverse event leading to hospitalization or prolongation of hospitalization will be considered as Serious, UNLESS at least one of the following expectations is met:
   The admission results in a hospital stay of less than 24 hours OR
   The admission is pre-planned (e.g., elective or scheduled surgery arranged prior to the start of the study) OR
   The admission is not associated with an adverse event (e.g., social hospitalization for purposes of respite care.
However, it should be noted that invasive treatment during any hospitalization may fulfill the criteria of “medically important” and as such may be reportable as a serious adverse event dependent on clinical judgment. In addition, where local regulatory authorities specifically require a more stringent definition, the local regulation takes precedent.
Results in persistent or significant disability/incapacity. The definition of disability is a substantial disruption of a person's ability to conduct normal life's functions.
Is a congenital anomaly/birth defect.
Is an important medical event. Important medical events that may not result death, be life-threatening, or require hospitalization may be considered a serious adverse experience when, based upon appropriate medical judgment, they may jeopardize the subject and may require medical or surgical intervention to prevent one of the outcomes listed in this definition. Examples of such medical events include allergic bronchospasm requiring intensive treatment in an emergency room or at home, blood disease or disorders, or convulsions that do not result in inpatient hospitalization, or the development of drug dependency or drug abuse. The development of a new cancer is always considered an important medical event.

Adverse Event Evaluation

The investigator or designee is responsible for ensuring that all adverse events (both serious and non-serious) observed by the clinical team or reported by the subject which occur after the subject has signed the informed consent are fully recorded in the subject's medical records. Source documentation must be available to support all adverse events.

A laboratory test abnormality considered clinically relevant (e.g., causing the subject to withdraw from the study, requiring treatment or causing apparent clinical manifestations, result in a delay or dose modification of study treatment, or judged relevant by the investigator), should be reported as an adverse event.

The investigator or sub-investigator (treating physician if applicable) will provide the following for all adverse events (both serious and non-serious):
Event term (as per CTCAE)
Description of the event
Date of onset and resolution
Expectedness of the toxicity
Grade of toxicity
Attribution of relatedness to the investigational agent-(this must be assigned by an investigator, sub-investigator, or treating physician)
Action taken as a result of the event, including but not limited to; no changes, dose interrupted, reduced, discontinued, etc. or action taken with regard to the event, i.e., no action, received conmed or other intervention, etc.
Outcome of event.

Descriptions and grading scales found in the NCI Common Terminology Criteria for Adverse Events (CTCAE) version 5.0 will be utilized for AE reporting.

An expected adverse event is an event previously known or anticipated to result from participation in the research study or any underlying disease, disorder, or condition of the subject. The event is usually listed in the Investigator Brochure, consent form or research protocol.

An unexpected adverse event is an adverse event not previously known or anticipated to result from the research study or any underlying disease, disorder, or condition of the subject.

Attribution is the relationship between an adverse event or serious adverse event and the study drug. Attribution will be assigned as follows:
Definite—The AE is clearly related to the study drug.
Probable—The AE is likely related to the study drug.
Possible—The AE may be related to the study drug.
Unlikely—The AE is doubtfully related to the study drug.
Unrelated—The AE is clearly NOT related to the study drug.

Protocol must specify if attribution is required for individual components of the treatment regimen or the treatment regimen as a whole.

### Devimistat

Product Description

Devimistat is provided in 10-mL amber glass vials. Each vial contains 10 mL of devimistat at a concentration 50 mg/mL, equivalent to 500 mg of devimistat. The drug product of devimistat is a clear and colorless solution that is free of any particulate matter.

Solution Preparation

Devimistat is to be given as an IV infusion at a rate of 4 mL/min via a central venous catheter on Day 1, prior to administration of mFOLFIRINOX. It is also given on Day 3, after completion of mFOLFIRINOX administration. On day 3, in the event that time will not permit devimistat administration following 5FU infusion, devimistat may be administered concurrently with 5FU via a double lumen port. The drug will be administered at a total dose of 500 mg/m² on day 1 and day 3, as described.

Devimistat must be diluted from 50 mg/mL to 12.5 mg/mL with 5% Dextrose Water or D5W (i.e., 1 portion of devimistat diluted with 3 portions of D5W) prior to administration. The diluted drug product should be visually inspected for clarity. If haziness, precipitate or coloration (other than colorless) is observed, do not use the diluted drug product for dosing. After dilution with sterile D5W, the solution is clear and has a pH of 8.4-8.8. The diluted devimistat drug product has been found to be stable for 24 hrs at room temperature and refrigeration temperature.

Devimistat must be administered IV, via an IV catheter that is free flowing and free of air in the dead space of the IV catheter, to minimize vascular irritation, inflammation and acute toxicity of devimistat (Study NCL-049). Accidental co-administration of extra air in the dead space of IV catheters during administration of devimistat has demonstrated the potential to induce acute toxicity of devimistat according to animal studies. Also, accidental leakage of devimistat into the perivascular space during IV administration, which prolongs exposure of perivascular tissue to devimistat, can induce significant local inflammation according to animal studies. To avoid local reactions at and around the site of administration, devimistat must be administered via a central venous catheter.

Devimistat must not be administered as a bolus, but by infusion, at a rate of ˜4 mL/min, via a central venous catheter with D5W running at a rate of about 125-150 mL/hr. This is to minimize potential acute toxicity of devimistat, according to animal studies.

The following precautions must be taken when administering devimistat:
Confirmation of the placement of the IV line to ensure a lack of leakage of devimistat into the perivascular space.
Confirmation that the IV line is free flowing.
Confirmation that the IV line is free of dead air space.
Dilute devimistat drug product with D5W, as instructed in the study protocol.
Administer devimistat by infusion, not as a bolus.
After administration of devimistat, flush the IV line with ˜10 mL of D5W to remove residual devimistat.
To avoid local reactions at and around the site of administration, devimistat should be administered via a central venous catheter.

Devimistat must be administered IV by infusion (˜4 mL/min), via an IV catheter with D5W running at a rate of about 125-150 mL/hr. To avoid local reactions at and around the site of administration, devimistat should be administered via a central venous catheter. Subsequent sections describe the appropriate types of IV catheters, IV bags, syringes and clinical solutions that can be used in mixing and administering devimistat to patients.

Devimistat can cause leaching of DEHP from IV infusion sets and IV bags. Therefore, DEHP-containing IV infusion sets, IV bags or syringes should not be used in mixing or administration of devimistat. Examples of the IV sets, IV bags and syringes that do not contains DEHP and therefore can be used in the administration of devimistat are:
Extension Set for Syringe Pump Use: All extension sets from MED-RX do not contain DEHP.
Syringes: Kendall Monoject syringes, all mono-ject syringes are DEHP free.

A compatibility study has been conducted showing that devimistat is compatible with 4 commonly used IV infusion sets. Therefore, these 4 types of IV infusion sets, and IV infusion sets that are made with the same materials, can be used to administer devimistat. These IV infusion sets are:
PVC material—ADDitIV® Primary IV Set with Universal Spike, Backcheck Valve, 2 Injection Sites, DEHP-Free and Latex-Free, 15 drops/mL, REF V14453, B Braun Medical Inc.
Latex material—Interlink® System Secondary Medication Set, 10 drops/mL, 2C7451, Baxter Healthcare Corporation
PVC material—Surshield™ Safety Winged Infusion Set, 0.19 mL Volume, Latex-Free, DEHP-Free, SV*S25BLS, Terumo Medical Products Hangzhou Co. Ltd.
Polyethylene material—Interlink® System Paclitaxel Set by Baxter HealthCare, NonDEHP-free: Polyethylene tubing with a 0.22 micro filter Item #2C7558 10 drops/mL (NOTE: devimistat does not require an in-line filter).

Compatibility studies have shown that devimistat drug product (50 mg/mL), and drug product diluted with D5W to various concentrations (1.6-25 mg/mL) are compatible with various types of syringes, as listed below. Therefore, any of these types of syringes, and syringes that are made with the same materials, can be used to administer devimistat. Also, glass syringes can also be used, since glass (such as glass containers) is compatible with devimistat drug product.
Norm-Ject, polyethylene barrel, polyethylene plunger, latex free (Henke Sass Wolf GMBH) syringes
Becton Dickinson syringes
Terumo syringes
Monoject syringes
Glass syringes

Devimistat drug product is slightly photosensitive. Therefore, after removal of devimistat drug product from the amber vials, devimistat drug product should be protected from excessive light before administration to patients. Devimistat should be stored under refrigeration, at 2°-8° C. (36°-46° F.), except when being prepared for administration.

Route of administration: 500 mg/m², IV infusion at a rate of 4 mL/min via a central venous port on days 1 and 3, every 2 weeks.

### Commercial Agents

Oxaliplatin

Other Names:

Oxaliplatin is commercially available under the tradename ELOXATIN.

Product Description:

Powder for solution for infusion: ELOXATIN is supplied in vials containing 50 mg or 100 mg of oxaliplatin as a sterile, preservative-free lyophilized powder for reconstitution. Lactose monohydrate is present as an inactive ingredient at 450 mg and 900 mg in the 50 mg and 100 mg dosage strengths, respectively.

Concentrate for solution for infusion: ELOXATIN is supplied in vials containing 50 mg, 100 mg or 200 mg of oxaliplatin as a sterile, preservative-free, aqueous solution at a concentration of 5 mg/mL.

ELOXATIN is incompatible in solution with alkaline medications or media (such as basic solutions of 5-fluorouracil) and must not be mixed with these or administered simultaneously through the same infusion line. The infusion line should be flushed with 5% Dextrose Injection, USP prior to administration of any concomitant medication. Parenteral drug products should be inspected visually for particulate matter and discoloration prior to administration and discarded if present.

Needles or intravenous administration sets containing aluminum parts that may come in contact with ELOXATIN should not be used for the preparation or mixing of the drug. Aluminum has been reported to cause degradation of platinum compounds.

Solution Preparation

Powder for solution for infusion: Reconstitution or final dilution must never be performed with a sodium chloride solution or other chloride containing solutions. The lyophilized powder is reconstituted by adding 10 mL (for the 50 mg vial) or 20 mL (for the 100 mg vial) of Water for Injection, USP or 5% Dextrose Injection, USP. Do not administer the reconstituted solution without further dilution. The reconstituted solution must be further diluted in an infusion solution of 250-500 mL of 5% Dextrose Injection, USP.

After reconstitution in the original vial, the solution may be stored up to 24 hours under refrigeration [2-8° C. (36-46° F.)]. After final dilution with 250-500 mL of 5% Dextrose Injection, USP, the shelf life is 6 hours at room temperature [20-25° C. (68-77° F.)] or up to 24 hours under refrigeration [2-8° C. (36-46° F.)].

ELOXATIN is not light sensitive. Store under normal lighting conditions at 25° C. (77° F.); excursions permitted to 15-30° C. (59-86° F.).

Concentrate for Solution for Infusion: Do not freeze and protect from light the concentrated solution. A final dilution must never be performed with a sodium chloride solution or other chloride containing solutions. The solution must be further diluted in an infusion solution of 250-500 mL of 5% Dextrose Injection, USP. Store at 25° C. (77° F.); excursions permitted to 15-30° C. (59-86° F.). Do not freeze and protect from light (keep in original outer carton).

After dilution with 250-500 mL of 5% Dextrose Injection, USP, the shelf life is 6 hours at room temperature [20-25° C. (68-77° F.)] or up to 24 hours under refrigeration [2-8° C. (36-46° F.)]. After final dilution, protection from light is not required.

Route of Administration:

Oxaliplatin will be administered as a 2-hr IV infusion.

Drug Procurement:

Oxaliplatin must be obtained from commercial sources

Folinic Acid

Folinic acid is also known as leucovorin. Folinic acid is commercially available as the calcium salt of folinic acid (i.e., calcium folinate), which has the name leucovorin calcium.

Product Description of Leucovorin Calcium:

Leucovorin Calcium Injection USP is a sterile, preservative-free solution indicated for intramuscular (IM) or intravenous (IV) administration in a 50 mL single-dose vial. Each mL contains leucovorin calcium equivalent to mg Leucovorin, USP; 8 mg sodium chloride; sodium hydroxide and/or hydrochloric acid for pH adjustment pH 7.8 (6.5 to 8.5). There is 0.004 mEq of calcium per mg of leucovorin. Solution contains no bacteriostat or antimicrobial agents. Leucovorin Calcium for Injection is a sterile product indicated for intramuscular (IM) or intravenous (IV) administration and is supplied in 50 mg, 100 mg, 200 mg, and 350 mg vials. Each 50 mg vial of Leucovorin Calcium for Injection, when reconstituted with 5 mL of sterile diluent, contains leucovorin (as the calcium salt) 10 mg/mL.

Solution Preparation:

Each 100 mg vial of Leucovorin Calcium for Injection, when reconstituted with 10 mL of sterile diluent, contains leucovorin (as the calcium salt) 10 mg/mL. Each 200 mg vial of Leucovorin Calcium for Injection, when reconstituted with 20 mL of sterile diluent, contains leucovorin (as the calcium salt) 10 mg/mL. Each 350 mg vial of Leucovorin Calcium for Injection, when reconstituted with 17.5 mL of sterile diluent, contains leucovorin (as the calcium salt) 20 mg/mL. In each dosage form, one milligram of leucovorin calcium contains 0.002 mmol of leucovorin and 0.002 mmol of calcium. These lyophilized products contain no preservative. The inactive ingredient is Sodium Chloride, USP, added to adjust tonicity. Reconstitute with Bacteriostatic Water for Injection, USP, which contains benzyl alcohol, or with Sterile Water for Injection, USP.

Storage Requirements:

Leucovorin Calcium Injection USP, 10 mg/mL, is supplied in sterile, single use vials as follows: NDC 55390-009-01 500 mg individually-boxed. Store in refrigerator 2° to 8° C. (36° to 46° F.). Protect from light. Discard unused portion. Retain in carton until time of use.

Leucovorin Calcium for Injection is supplied in sterile, single use vials as follows:
NDC 55390-051-10 50 mg boxed vial, packs of 10
NDC 55390-052-10 100 mg boxed vial, packs of 10.
NDC 55390-053-01 200 mg boxed vial. NDC 55390-054-01 350 mg boxed vial.

Store at 20° C. to 25° C. (68° to 77° F.). Protect from light. Retain in carton until time of use.

Route of Administration:

Folinic acid will be administered as a 90-min infusion immediately after oxaliplatin

Drug Procurement:

Folinic acid must be obtained by commercial sources.

Irinotecan

Irinotecan is commercially available in the form of a monohydrochloride, trihydrate salt, which is marketed under the tradename COMPTOSAR.

Product Description:

Irinotecan monohydrochloride, trihydrate that is marketed under the tradename CAMPTOSAR is supplied as a sterile, pale yellow, clear, aqueous solution. Each milliliter of solution contains 20 mg of irinotecan hydrochloride, trihydrate, 45 mg of Reference ID: 3674813 24 sorbitol, NF, and 0.9 mg of lactic acid, USP. The pH of the solution has been adjusted to 3.5 (range, 3.0 to 3.8) with sodium hydroxide or hydrochloric acid. CAMPTOSAR is intended for dilution with 5% Dextrose Injection, USP (D5W), or 0.9% Sodium Chloride Injection, USP, prior to intravenous infusion. The preferred diluent is 5% Dextrose Injection, USP.

Solution Preparation:

Inspect vial contents for particulate matter and discoloration and repeat inspection when drug product is withdrawn from vial into syringe.

CAMPTOSAR Injection 20 mg/mL is intended for single use only and any unused portion should be discarded.

CAMPTOSAR Injection must be diluted prior to infusion. CAMPTOSAR should be diluted in 5% Dextrose Injection, USP, (preferred) or 0.9% Sodium Chloride Injection, USP, to a final concentration range of 0.12 mg/mL to 2.8 mg/mL. Other drugs should not be added to the infusion solution.

The solution is physically and chemically stable for up to 24 hours at room temperature and in ambient fluorescent lighting. Solutions diluted in 5% Dextrose Injection, USP, and stored at refrigerated temperatures (approximately 2° to 8° C., 36° to 46° F.), and protected from light are physically and chemically stable for 48 hours. Refrigeration of admixtures using 0.9% Sodium Chloride Injection, USP, is not recommended due to a low and sporadic incidence of visible particulates. Freezing CAMPTOSAR and admixtures of CAMPTOSAR may result in precipitation of the drug and should be avoided.

The CAMPTOSAR Injection solution should be used immediately after reconstitution as it contains no antibacterial preservative. Because of possible microbial contamination during dilution, it is advisable to use the admixture prepared with 5% Dextrose Injection, USP, within 24 hours if refrigerated (2° to 8° C., 36° to 46° F.). In the case of admixtures prepared with 5% Dextrose Injection, USP, or Sodium Chloride Injection, USP, the solutions should be used within 4 hours if kept at room temperature. If reconstitution and dilution are performed under strict aseptic conditions (e.g., on Laminar Air Flow bench),

CAMPTOSAR Injection solution should be used (infusion completed) within 12 hours at room temperature or 24 hours if refrigerated (2° to 8° C., 36° to 46° F.).

Care should be exercised in the handling and preparation of infusion solutions prepared from CAMPTOSAR Injection. The use of gloves is recommended. If a solution of CAMPTOSAR contacts the skin, wash the skin immediately and thoroughly with soap and water. If CAMPTOSAR contacts the mucous membranes, flush thoroughly with water. Several published guidelines for handling and disposal of anticancer agents are available.

Storage Requirements:

Store at controlled room temperature 15° to 30° C. (59° to 86° F.). Protect from light. Keep the vial in the carton until the time of use. Inspect the vial for damage and visible signs of leaks before removing from the carton. If damaged, incinerate the unopened package.

Route of Administration:

Irinotecan will be administered as a 90-min IV infusion via a Y connector.

Drug Procurement:

Irinotecan must be obtained by commercial sources.

5-Fluorouracil

Other Names: 5-FU

Product Description:

Fluorouracil injection is supplied in a pharmacy bulk package available in a box containing one vial: NDC 68152-106-06: one box with one vial, containing 2.5 g/50 mL (50 mg/mL) fluorouracil.

Solution Preparation:

Fluorouracil is supplied in a pharmacy bulk package consisting of a vial. The pharmacy bulk package can be used to prepare doses for more than one patient. It is not supplied with a sterile transfer device, which is required for dispensing when multiple doses will be prepared from the single vial. The 50 mL vial is only intended for preparation in a Pharmacy Admixture Service under appropriate conditions for cytotoxic drugs. Store vial at room temperature. Using aseptic conditions, penetrate the container closure once with a suitable sterile transfer device or dispensing set that allows measured distribution of the contents. Record the date and time the vial was opened on the vial label. Discard the pharmacy bulk package 4 hours after penetration of the container closure. Withdraw the calculated dose for an individual patient into a sterile syringe. Inspect the solution in syringe for particulate matter and discoloration prior to administration or further dilution. Discard syringe if the solution is discolored or contains particulate matter. Dilute in D5W only.

Storage Requirements:

Storage Store at 20° C. to 25° C. (68° F. to 77° F.). Excursions are permitted from 15° C. to 30° C. (59° F. to 86° F.) [see USP Controlled Room Temperature]. Protect from light. Retain in carton until time of use. Fluorouracil is a cytotoxic drug. Follow applicable special handling and disposable procedures.

Route of Administration:

Fluorouracil (5FU) will be administered as bolus followed by a 46-hr infusion at 2400 mg/m², starting immediately after completion of folinic acid and irinotecan.

Exploratory or Correlative Studies

Analyses of Plasma Devimistat Concentrations, Metabolite Concentrations, and Other Analyses

Plasma samples for the determination of devimistat concentrations, metabolite concentrations and other analyses will be obtained before and at various times after dosing of devimistat will be conducted in the first 15 patients. This includes:
Day 1 of Cycle 1 at the following time points: −5 min before dosing, and at 0.5, 1, 1.5, 2 & 24 hours after the first dose of devimistat.
Day 3 of Cycle 1, immediately prior to the administration of devimistat.
Day 4 of Cycle 1, immediately prior to the administration of Neulasta, if applicable.

Blood (4 mL for each sample) should be obtained from an IV catheter different from the one that is used to administer devimistat. The blood samples should be collected in 4-mL lavender top collection tubes containing K2-EDTA anticoagulant. The procedures for obtaining plasma samples are described in subsequent sections.

Devimistat and Metabolite Assays

Plasma concentrations of devimistat and possibly metabolites will be assayed using the validated Liquid Chromatography (LC)-Mass Spectroscopy/Mass Spectroscopy (LC/MS/MS) method (Rafael Study #VLD-002). Individual and mean plasma concentrations of devimistat and possibly metabolites for each patient will be tabulated at each time point and presented graphically. The following parameters will be estimated in all patients (as appropriate) from individual plasma concentration vs. time data:
the maximum observed concentration (Cₘₐₓ)
area under the curve from time zero till the last measurable concentration (AUC₀₋ₜ, calculated by the linear trapezoidal rule and extrapolated to infinity by acceptable methods)
area under the curve from time zero to infinity (AUC_{0-infinity})
Kₑₗ (estimated by linear regression of the terminal elimination phase)
t_{1/2}
Cl
V_{d}
C_{end}

Correlative Studies—Other Analyses

The following samples will be collected at screening (or C1D1), and then every 4^{th} cycle during the 6 months of treatment (at cycles 4, 8, and 12). Following treatment, these samples will be collected at 3 months following treatment and then 6 months following treatment and at progression following physician exam:
Green top tubes (sodium heparin) −20 mL (2 tubes)
Lavender top tubes (EDTA) −3 tubes×4 mL each
Paxgene tube (DNA) −2.5 mL.

Tissue Specimen Processing

Standard paraffin embedded tissue obtained for routine clinical use including tissue obtained for diagnostic biopsies or removed during surgery will be analyzed for biomarkers of outcome and treatment response.

### Study Parameters

Study Procedures/Evaluations
Medical history will be obtained by interview
Medications will be obtained by interview.
Physical examination will be a full exam including HEENT and Neck, Chest and Lungs, Cardiovascular, Abdomen, GU, Musculoskeletal, Neurological and Extremities/Skin systems.
Imaging: Pancreas protocol CT scan includes: a dedicated 64 channel, multi-phase, pancreas protocol CT examination with water given orally. Patients undergo an injection at a rate of 4 ml/s of 100-120 ml non-ionic iodinated (300-320 mg I/ml) contrast material (Optiray 320, Covidien, Mansfield, MA or Ultravist 300, Bayer Healthcare, Wayne, NJ). Acquisition phases in chronological order include unenhanced, early arterial (arteriogram), late arterial (pancreatic parenchymal) and venous phases. Post-contrast phases are acquired at 0, 10 and 35 seconds after bolus arrival to the aorta at the mid-liver level. For all phases, 0.6-0.75 mm thick axial images are obtained. Images are reconstructed at: 5 mm for the unenhanced phase, 3 mm for the early arterial phase, 3 mm and 1.5 mm for the later arterial phase, and 3 mm for the venous phase. Early arterial and venous phase images are reconstructed at the scanner consoles using multi-planar reformatting and 3D surface rendering for enhanced vascular anatomy visualization (Fong Z V, Tan W P, Lavu H, Kennedy E P, Mitchell D G, Koniaris L G, et al, “Preoperative imaging for resectable periampullary cancer: clinicopathologic implications of reported radiographic findings,” Journal of Gastrointestinal Surgery: Official Journal of the Society for Surgery of the Alimentary Tract, 2013, 17(6), 1098-106). If a patient has an allergy to contrast, imaging studies without contrast may be performed instead.
Tissue will be obtained for clinical purposes and stored as paraffin embedded blocks. These will be diagnostic biopsies or surgical specimens for patients undergoing resection.
Routine clinical laboratory tests will be performed according to standard procedures.
FACT-Hep quality of life questionnaires will be obtained in person or by telephone contact by trained research staff every 3 months for the first year of the follow-up period. A numerical value MUST be entered for each item on the questionnaire. Not applicable (N/A) is NOT an option for a response on this validated questionnaire and cannot be used as an answer to a question. If the patient prefers not to answer the sexual activity question then the box next to the question is required to be checked.

Clinical Laboratory Evaluations

Clinical Chemistry assessment includes: Glucose, BUN, Creatinine, AST/serum glutamic-oxaloacetic transaminase (SGOT), Total protein, ALT/serum glutamic-pyruvic transaminase (SGPT), Albumin Alkaline phosphatase (ALP), Nat, Total bilirubin, K⁺, CEA, Cl⁻, CA-19-9, Mg, Ca²⁺, P (phosphorus), HCO³⁻ (bicarbonate).

Hematology includes: Complete blood count, Differential count, Platelet count, hemoglobin, hematocrit.

Coagulation includes: Prothrombin time, Partial thromboplastin time.

### Measurement of Effect

After the first four cycles (approximately two months) of treatment with devimistat+mFOLFIRINOX, tumor response will be assessed using the same modalities of imaging used at screening. Serum levels of relevant tumor markers CEA and/or CA 19-9 will be drawn, and performance status determined. RECIST criteria in combination with the clinical judgement of the PI and treating medical oncologist will be used to determine whether additional courses of therapy are appropriate; if progression is noted, the patient will be removed from the study. CT chest with IV contrast and a pancreas protocol CT are preferred when possible, although an MRI of the abdomen and pelvis with contrast may also be obtained as an alternative.

Should the medical oncologist and principal investigator determine that the patient has stable disease or has had a favorable response, those patients will continue treatment with devimistat+mFOLFIRINOX for an additional four cycles (approximately two months). At that time, patients will then undergo reimaging of the chest, abdomen, and pelvis. Serum levels of tumor markers CEA and/or CA 19-9, and performance status will be reevaluated. Again, patients who are determined by the principal investigator to have progressed will be removed from the study, while those who are determined to have stable disease or favorable response will continue, with subsequent re-imaging after another four cycles, unless unacceptable toxicity or progression is encountered.

Assessment of tumor response will be the responsibility of the principal investigator and should be based on radiologic findings, as performed directly by a Research Review radiologist. There will be a separate Research Review by a radiologist aside from the clinical review, and the Research Review will be used to determine progression.

### Statistical Considerations

Analysis Plans

Primary Outcome Analysis

The primary outcome for this study is overall survival. Survival time will be calculated from the date of enrollment. This is a single-arm study with comparison to historical data. The median OS with locally advanced PDA based on the phase II and III data and institutional data is around 15 months (Conroy, T et al., 2011 cited above). Data specific for FOLFIRINOX range widely in the literature and cannot be used to extrapolate easily because many of these studies involved heterogeneous populations from retrospective studies, groups that are not precisely defined as locally advanced with rigorous radiologic review, or are highly selected with very favorable results. For instance, the median OS in 36 patients in a Canadian study was 23 months (Chllamma, M K et al, 2016) and as high as 26 months in 31 patients in a separate study from Yale (Stein, S M et al., 2016). Overall survival in a similarly sized cohort from Japan was just 9 months (Muranaka T, Kuwatani M, Komatsu Y, Sawada K, Nakatsumi H, Kawamoto Y, et al, “Comparison of efficacy and toxicity of FOLFIRINOX and gemcitabine with nabpaclitaxel in unresectable pancreatic cancer,” Journal of Gastrointestinal Oncology, 2017, 8(3), 566-71). These small studies are difficult to generalize due to their sample size. In our own experience of 69 patients, the overall survival was 14 months in patients with locally advanced PDA and was similar in both borderline and unresectable patients. Additionally, patients receiving FOLFIRINOX in this population had a median survival of only 14 months (for both borderline and unresectable patients), suggesting perhaps more advanced disease or aggressive biology in our specific patient population. Thus, we will use a median 15 month OS as a historical benchmark. The null hypothesis to be tested is that the median overall survival time is less than or equal to 15 months vs. the alternative that it is greater than 15 months. The median survival time will be estimated using the Kaplan-Meier method along with a two-sided 80% confidence interval. If the lower bound of the confidence interval is greater than 15 months, the null hypothesis will be rejected. The analysis will be performed 12 months after the last patient has accrued (expected at 24 months+12 months), but we will continue to follow patients out for 3 years as part of a long-term survivorship plan.

Secondary Outcome Analysis

The distribution of PFS and time to progression will be estimated using the Kaplan-Meier method. Progression free survival time is defined as time from enrollment until progression or death. Patients alive at the end of follow-up are censored. Time to progression is similar to PFS, but censors patients who die but have not progressed. Median PFS and TTP will be estimated with an exact 95% binomial confidence interval. The rate of negative resection margins and complete resection rate will be estimated as well with 95% confidence intervals.

Correlative Outcome Analysis

Correlative endpoints will be summarized by response status.

Analysis Cohorts

The primary analysis cohort will include all study subjects who receive at least 2 months of therapy. Subset analyses may be performed in an exploratory fashion. All subjects will be evaluated for safety.

Sample Size Considerations

Assuming an accrual rate of 30 patients per year (achievable based on historical experience), and a 12 month follow-up time, and a one-sided alpha of 0.10, a sample size of 30 patients, accrued over 24 months provides 81.3% power to detect an increase in median survival from 15 to 28 months. Allowing for possible patient to drop-out before completion of 2 months of therapy, we will recruit 33 patients. The hazard ratio is 0.54. Subjects will not be replaced.

Evaluation of Safety

Grade 3 and 4 toxicity rates of devimistat and mFOLFIRINOX have been published (Alistar A, Morris B B, Desnoyer R, Klepin H D, Hosseinzadeh K, Clark C, et al, “Safety and tolerability of the first-in-class agent CPI-613 in combination with modified FOLFIRINOX in patients with metastatic pancreatic cancer: a single-centre, open-label, dose-escalation, phase 1 trial,” Lancet Oncol, 2017,18(6), 770-8). Toxicities form observed in this trial will be tabulated in a descriptive fashion and compared to historical rates.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for treating pancreatic cancer in a patient in need thereof, comprising the step of intravenously administering to the patient:
a. devimistat,
b. oxaliplatin,
c. folinic acid,
d. irinotecan, and
e. fluorouracil
pursuant to a treatment cycle of 14 days, wherein during the treatment cycle the devimistat is intravenously administered on each of days 1 and 3 at a daily dose of about 750 mg/m² or about 1,000 mg/m², the oxaliplatin is intravenously administered on day 1 as a single dose of about 65 mg/m², the folinic acid is intravenously administered on day 1 as a single dose of about 400 mg/m², the irinotecan is intravenously administered on day 1 as a single dose of about 121 mg/m², and the fluorouracil is intravenously administered on day 1 as a bolus of about 400 mg/m² followed by an infusion of about 2,400 mg/m² for about 42-48 hours over days 1, 2, and 3, in order to treat the pancreatic cancer.

2. (canceled)

3. (canceled)

4. The method of claim 1, wherein the pancreatic cancer is metastatic pancreatic cancer.

5. The method of claim 1, wherein the pancreatic cancer is locally advanced.

6. The method of claim 1, wherein the pancreatic cancer is locally advanced pancreatic ductal adenocarcinoma.

7. The method of claim 1, wherein the during the treatment cycle the devimistat is intravenously administered at a daily dose of about 750 mg/m² on each of days 1 and 3.

8. The method of claim 1, wherein the during the treatment cycle the devimistat is intravenously administered at a daily dose of about 1000 mg/m² on each of days 1 and 3.

9. The method of claim 1, wherein the treatment cycle is repeated one or more times.

10. The method of claim 1, wherein each devimistat dose is administered intravenously to the patient as a solution containing about 12.5 mg/mL of devimistat.

11. The method of claim 1, wherein each devimistat dose is administered intravenously to the patient as a solution containing triethanolamine and about 12.5 mg/mL of devimistat.

12. The method of claim 1, wherein each devimistat dose is administered intravenously to the patient as a solution containing water, triethanolamine, and about 12.5 mg/mL of devimistat.

13. The method of claim 1, wherein each devimistat dose is administered intravenously to the patient as a solution containing dextrose, water, triethanolamine, and about 12.5 mg/mL of devimistat.

14. The method of claim 1, wherein each devimistat dose is administered intravenously to the patient as a solution containing water, about 0.25 M triethanolamine, and about 12.5 mg/mL of devimistat.

15. The method of claim 1, wherein each devimistat dose is administered intravenously to the patient as a solution containing water, dextrose, about 0.25 M triethanolamine, and about 12.5 mg/mL of devimistat.

16. The method of claim 10, wherein the solution containing about 12.5 mg/mL of devimistat is prepared by the steps of:
a. providing a 50 mg/mL solution of devimistat in 1 M aqueous triethanolamine; and
b. diluting the 50 mg/mL solution with sterile 5% dextrose for injection (D5W) to a concentration of about 12.5 mg/mL.

17. The method of claim 1, wherein the folinic acid is administered as an alkaline earth metal salt of folinic acid.

18. The method of claim 17, wherein the alkaline earth metal salt of folinic acid is calcium folinate.

19. The method of claim 1, wherein the irinotecan is administered as irinotecan hydrochloride.

20. The method of claim 1, wherein the irinotecan is administered as irinotecan hydrochloride trihydrate, and the dose of irinotecan hydrochloride trihydrate is about 140 mg/m².

21. (canceled)
